(19) United States
(12) Patent Application Publication
(10) Pub. No.: US 2026/0256879 A1
(43) Pub. Date: Sep. 3, 2026

(54) COMBINATION THERAPY WITH MEK INHIBITORS FOR THE TREATMENT OF CANCER

(71) Applicants: FUNDACIÓ PRIVADA INSTITUT D'INVESTIGACIÓ ONCOLÒGICA DE VALL HEBRON, Barcelona (ES); INSTITUCIÓ CATALANA DE RECERCA I ESTUDIS AVANÇATS, Barcelona (ES)

(72) Inventors: Mariano Federico ZACARIAS FLUCK, Barcelona (ES); Jastrinjan KAUR, Biel/Bienne (CH); Laura SOUCEK, Barcelona (ES)

(21) Appl. No.: 19/491,214

(22) PCT Filed: Jun. 6, 2024
(86) PCT No.: PCT/EP2024/065522

§ 371 Date: Dec. 8, 2025

(30) Foreign Application Priority Data
Jun. 7, 2023 (EP) 23382553.8

(51) Int. Cl. A61K 38/17; A61K 31/4184; A61K 31/44; A61K 31/519; A61P 35/00

(52) U.S. Cl. CPC A61K 38/1709; A61K 31/4184; A61K 31/44; A61K 31/519; A61P 35/00

(57) ABSTRACT

The invention relates to a combination of a MEK inhibitor with Omomyc, a functionally equivalent variant thereof, a conjugate comprising Omomyc or said functionally equivalent variant, a polynucleotide encoding said polypeptides, a vector comprising said polynucleotide and a cell capable of secreting the polypeptide or the conjugate. The invention also relates to pharmaceutical compositions containing the combination of the invention and to their medical uses, particularly their uses in the treatment of cancer.

### FIELD OF THE INVENTION

The present invention relates to the field of cancer and, more particularly, to a combination comprising mitogen-activated protein kinase kinase (MEK) inhibitors and Omomyc and its use in medicine, more particularly in the prevention and/or treatment of cancer.

### BACKGROUND OF THE INVENTION

Cancer is a leading cause of death worldwide, accounting for nearly 10 million deaths in 2020. It is a large subset of diseases characterized by the uncontrollable growth of abnormal cells.

The mitogen-activated protein kinase (MAPK) cascade is an intracellular signaling pathway involved in the regulation of cellular proliferation and the survival of tumor cells. Several different mutations involving BRAF (v-raf murine sarcoma viral oncogene homolog 1) or NRAS (neuroblastoma RAS viral oncogene homolog) exert an oncogenic effect by activating the MAPK pathway. MEK is downstream of BRAF in the MAPK signaling pathway. Since mutations in both NRAS and BRAF signal through MEK, inhibition of MEK has proven to be an attractive therapeutic strategy for cancers carrying BRAF and NRAS activating mutations.

Melanoma is an aggressive form of skin cancer arising from malignantly transformed melanocytes whose incidence and mortality is rising. Long term survival in patients with metastatic melanoma is <10% and their median overall survival is <1 year. This emphasizes the need to develop novel, effective and more individualized treatments for metastatic melanoma patients.

Activation of the MAPK signaling pathway by oncogenic mutations has been found in up to 90% of melanoma cases. The most common oncogene to be mutated in the MAPK signaling pathway in melanoma is BRAF. However, 30% of all melanoma activating MAPK pathway harbor mutations in NRAS. Therefore, melanoma has been treated with MEK inhibitors.

Four MEK inhibitors (trametinib, binimetinib, selumetinib and cobimetinib) have been approved by the United States Food and Drug Administration (FDA). However, MEK inhibitor's monotherapy seem to have poor clinical outcomes and more toxicities compared with chemotherapy alone. Therefore, combined therapies with other drugs have been developed.

Trametinib is used as a first-line treatment for metastatic melanoma patients carrying the BRAF and NRAS mutations in combination with BRAF inhibitors.

Unfortunately, most patients sensitive to the initial treatment with these drugs eventually acquire resistance to the combined treatment of BRAF inhibitors with inhibitors of downstream MAPK pathway targeting MEK. This underlines the need for more effective and combinatorial treatments to overcome these limitations.

Therefore, there is still a need in the state of the art to develop novel and improved therapeutic approaches for the treatment of cancer that can overcome resistance to MEK inhibitors increasing its potency and/or reducing their adverse effects.

### BRIEF SUMMARY OF THE INVENTION

In a first aspect, the invention relates to a combination comprising:
i) a first component selected from the group consisting of:
   a) a polypeptide comprising the sequence SEQ ID NO: 1 or a functionally equivalent variant thereof;
   b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof;
   c) a polynucleotide encoding the polypeptide of a) or the conjugate of b);
   d) a vector comprising the polynucleotide according to c); and
   e) a cell capable of secreting into the medium the polypeptide according to a) or the conjugate according to b);
and
ii) a second component that is a MEK inhibitor.

In a second aspect, the invention relates to a pharmaceutical composition comprising a pharmaceutically effective amount of a combination according to the invention and a pharmaceutically acceptable excipient.

In a third aspect, the invention relates to a combination according to the invention or a pharmaceutical composition according to the invention for use in medicine.

In a fourth aspect, the invention relates to a combination according to the invention or a pharmaceutical composition according to the invention for use in the prevention and/or treatment of cancer.

### DESCRIPTION OF THE FIGURES

FIG. 1. Bar-charts representing synergistic combinations between Omomyc and trametinib for two melanoma cell lines. (A) SkMel28 and (B) SkMel37 treated with either Omomyc, Trametinib or their combination at the indicated concentrations for 5 days. Relative cell number was inferred by AlamarBlue viability test.

FIG. 2. Bar-charts representing synergistic combinations between Omomyc and selumetinib for two melanoma cell lines. (A) SkMel28 and (B) SkMel37 treated with either Omomyc, selumetinib or their combination at the indicated concentrations for 5 days. Relative cell number was inferred by AlamarBlue viability test.

FIG. 3. Bar-charts representing synergistic combinations between Omomyc and pimasertib for two melanoma cell lines. (A) SkMel28 and (B) SkMel37 treated with either Omomyc, pimasertib or their combination at the indicated concentrations for 5 days. Relative cell number was inferred by AlamarBlue viability test.

### DETAILED DESCRIPTION OF THE INVENTION

The present invention relates to the provision of new therapeutic combinations for the prevention and treatment of cancer.

Unless otherwise defined, all technical terms used herein have the same meaning as commonly understood by one of ordinary skill in the art to which this invention belongs.

All the embodiments disclosed in relation to an aspect of the invention are applicable to the other aspects.

### Combinations and Pharmaceutical Compositions of the Invention

The definitions provided herewith and in every other aspect of the invention are equally applicable to the whole invention.

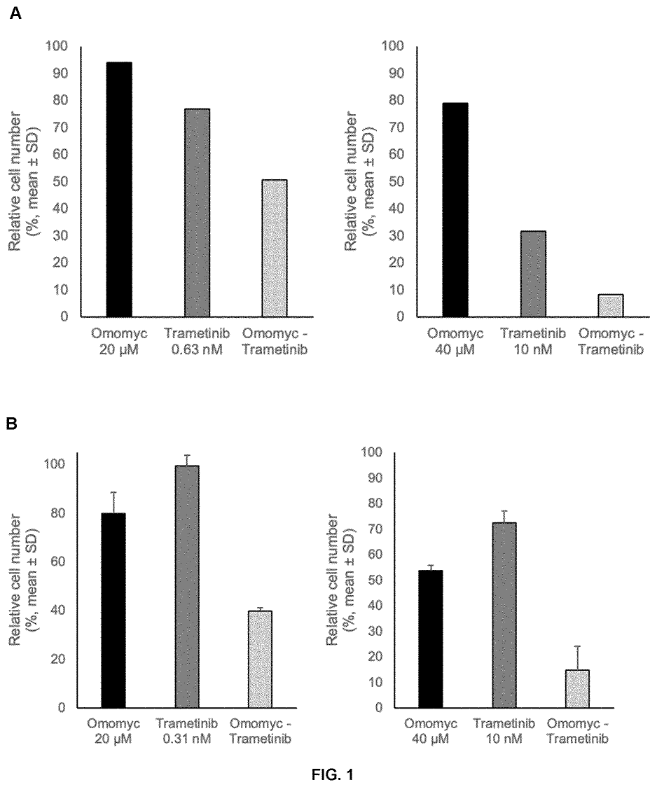

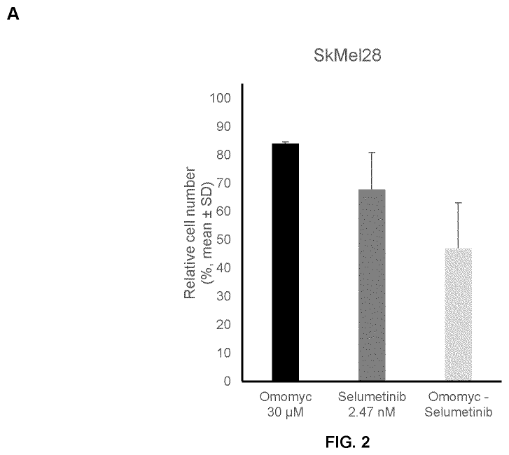

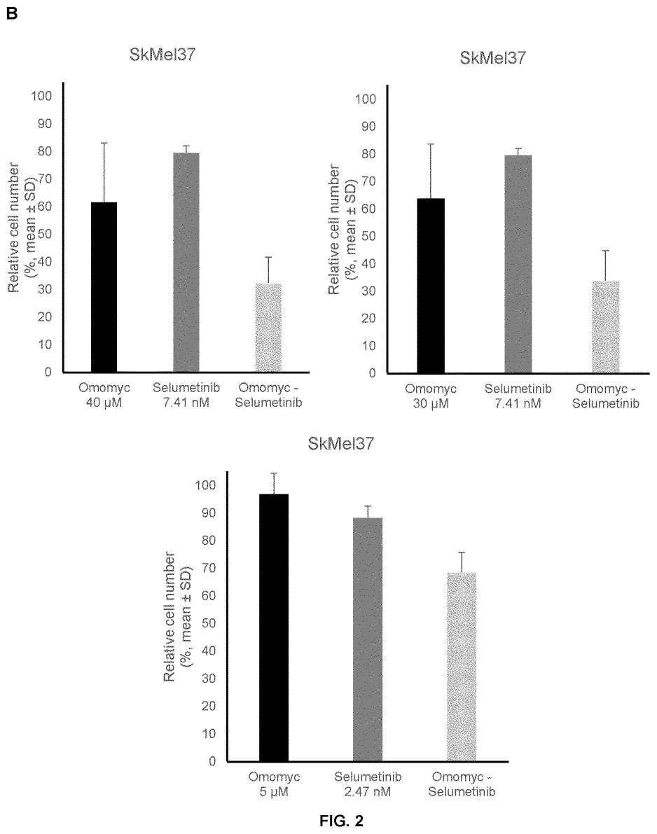

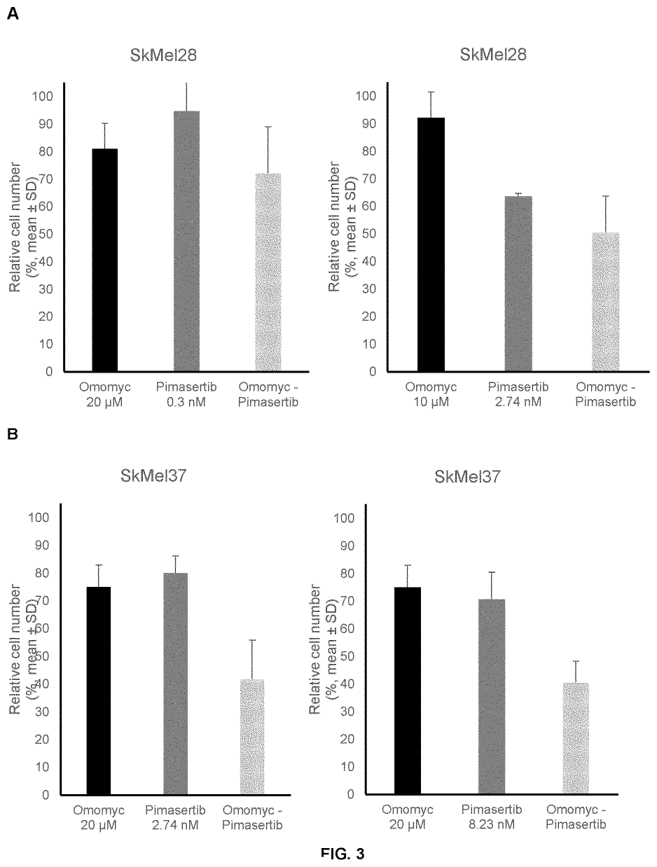

The authors of the present invention have surprisingly found that the combination of Omomyc and a MEK inhibitor has a synergistic effect in treating cancers. The authors have shown that a combination of Omomyc and the MEK inhibitor trametinib synergistically decreases the viability of melanoma cell lines SkMel28 (FIG. 1A) and SkMel37 (FIG. 1B). Combinations of Omomyc and selumetinib (FIG. 2) and Omomyc and pimasertib (FIG. 3) also decrease synergistically the viability of SkMel28 and SKmel37 cell lines. This synergistic effect is maintained regardless the dose resulting in a beneficial effect, in particular, an increase in the therapeutic effect of the composition of the invention relative to each of its components so that it can reach the same result with lower doses of each of the components, thereby reducing the side effects on the subject receiving the composition of the invention.

Omomyc can also be useful to revert resistance to MEK inhibitors by enhancing sensitivity to them. The combination of the invention broadens the population who may be responsive to a therapy based on MEK inhibitors.

Therefore, a combination of Omomyc with a MEK inhibitor can be an effective therapy for the treatment of both BRAF-mutated and wild-type tumors, and for treating tumors resistant to MEK inhibitors.

Thus, in a first aspect, the invention relates to a combination comprising:
i) a first component selected from the group consisting of:
   a) a polypeptide comprising the sequence SEQ ID NO: 1 or a functionally equivalent variant thereof;
   b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof;
   c) a polynucleotide encoding the polypeptide of a) or the conjugate of b);
   d) a vector comprising the polynucleotide according to c); and
   e) a cell capable of secreting into the medium the polypeptide according to a) or the conjugate according to b);
and
ii) a second component that is a MEK inhibitor.

According to the invention, the expression “combination” stands for the various combinations of compounds (i) and (ii), for example in a composition formulated as a single formulation, in a combined mixture composed from separate formulations of each of the components, such as a “tank-mix” which may be combined for joint use as a combined preparation, and in a combined use of the single active ingredients when applied in a sequential manner, i.e., one after the other with a reasonably short period, such as a few hours or days or in simultaneous administration. In the present invention, compound (i) refers to a therapeutically effective amount of a polypeptide comprising the sequence SEQ ID NO: 1 or a functionally equivalent variant thereof, or refers to a conjugate comprising a polypeptide comprising the sequence SEQ ID NO: 1 or a functionally equivalent variant thereof and a chemical moiety that facilitates cellular uptake of the polypeptide or of the functionally equivalent variant thereof, or refers to a polynucleotide encoding the polypeptide or the conjugate, or refers to a vector comprising the polynucleotide, or refers to a cell capable of secreting into the medium the polypeptide or the conjugate. In the present invention, compound (ii) refers to a therapeutically effective amount of a MEK inhibitor. Preferably, the order of applying the compounds (i) and (ii) is not essential for working the present invention.

The combination may be a kit-of-parts wherein each of the components is individually formulated and packaged.

A combination of compounds (i) and (ii) can be formulated for its simultaneous, separate or sequential administration. Particularly, if the administration is not simultaneous, the compounds are administered in a close time proximity to each other. Furthermore, compounds are administered in the same or different dosage form or by the same or different administration route, e.g. one compound can be administered orally and the other compound can be administered intravenously. Preferably, compound (i) is administered intravenously and compound (ii) is administered orally. In another embodiment compound (i) is administered intranasally and compound (ii) is administered orally. In another embodiment, compounds (i) and (ii) are administered intravenously.

The combination of the two compounds (i) and (ii) can be administered:
as a combination that is being part of the same medicament formulation, the two compounds being then administered always simultaneously.
as a combination of two units, each with one of the substances giving rise to the possibility of simultaneous, sequential or separate administration.

In a particular embodiment, compound (i) of the combination of the invention is independently administered from compound (ii), i.e. in two units, but at the same time.

In another particular embodiment, compound (i) of the combination of the invention is administered first, and then compound (ii), i.e. the compound (ii) is separately or sequentially administered.

In yet another particular embodiment, compound (ii) of the combination of the invention is administered first, and then compound (i), i.e. the compound (i) is administered separately or sequentially, as defined.

If administered separately, compounds (i) and (ii) of the combination of the invention can be administered within a period of time from one another, for example, within 1, 2, 3, 4, 5, 6, 7, 8, 9, 10, 11, 12, 13, 14, 15, 16, 17, 18, 19, 20, 21, 22, 23, or 24 hours from one another. In another embodiment, compounds (i) and (ii) of the combination of the invention can be administered within 1, 2, 3, 4, 5, 6, 7, 8, 9, 10, 11, 12, 13, 14, 15, 16, 17, 18, 19, 20, 21, 22, 23, or 24 days from one another, preferably within 1 days from one another, more preferably within 10 days from one another. In a preferred embodiment, compound (ii) is administered after 10 days of the first administration of compound (i). In an embodiment the administration of the first compound is discontinued before starting the administration of the second compound.

In another aspect, the invention relates to a combination or pharmaceutical composition comprising a synergistically effective amount of a first component according to the first aspect of the invention and a MEK inhibitor.

### Compound (i) of the Combination of the Invention

In a preferred embodiment, compound (i) of the invention is a polypeptide comprising the sequence SEQ ID NO: 1 or a functionally equivalent variant thereof; more preferably a polypeptide comprising the sequence SEQ ID NO: 1.

The terms “polypeptide” and “peptide” are used interchangeably herein to refer to polymers of amino acids of any length. The polypeptide of the invention can comprise modified amino acids, and it can be interrupted by non-amino acids. In a preferred embodiment the polypeptide is exclusively formed by amino acids. Preferably the polypeptide that forms item (i) of the combination has a length between 80 and 500 amino acids, more preferably between 80 and 300 amino acids, more preferably between 80 and 250 amino acids, more preferably between 80 and 150, even more preferably between 80 and 130 amino acids, preferably between 90 and 130 amino acids, preferably no more than 125 amino acids, more preferably no more than 100 amino acids. In a preferred embodiment, the polypeptide has a length between 90 and 98 amino acids, preferably between 90 and 95 amino acids, more preferably 91 amino acids.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers). Preferably, the amino acids are L-amino acids.

The term “natural amino acids” or “naturally occurring amino acids” comprises the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

As used herein, the term “non-natural amino acid” or “synthetic amino acid” refers to a carboxylic acid, or a derivative thereof, substituted at position “a” with an amine group and being structurally related to a natural amino acid. Illustrative non-limiting examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxy lysine, alio hydroxy lysine, 3-hydroxyproline, 4-hydroxyproline, isodesmosine, alloisoleucine, N-methylglycine, N-methylisoleucine, 6-N-methyl-lysine, N-methylvaline, norvaline, norleucine, ornithine, etc.

The polypeptide of the present invention may also comprise non-amino acid moieties, such as for example, hydrophobic moieties (various linear, branched, cyclic, polycyclic or heterocyclic hydrocarbons and hydrocarbon derivatives) attached to the peptides; various protecting groups which are attached to the compound's terminals to decrease degradation.

Suitable protecting functional groups are described in Green and Wuts, “Protecting Groups in Organic Synthesis”, John Wiley and Sons, Chapters 5 and 7, 1991.

Chemical (non-amino acid) groups present in the polypeptide may be included in order to improve various physiological properties such as decreased degradation or clearance; decreased repulsion by various cellular pumps, improve various modes of administration, increased specificity, increased affinity, increased stability, bioavailability, solubility, decreased toxicity and the like.

“Mimetic” include molecules which mimic the chemical structure of a peptidic structure and retain the functional properties of the peptidic structure. Approaches to designing peptide analogs, derivatives and mimetics are known in the art.

In an embodiment the polypeptide of the invention is a polypeptide consisting of sequence SEQ ID NO: 1 or a polypeptide consisting of a functionally equivalent variant of SEQ ID NO: 1, preferably is a polypeptide consisting of the sequence SEQ ID NO: 1.

The SEQ ID NO: 1 corresponds to

(SEQ ID NO: 1)
TEENVKRRTHNVLERQRRNELKRSFFALRDQIPELENNEKAPKVVILKK
ATAYILSVQAETQKLISEIDLLRKQNEQLKHKLEQLRNSCA

The polypeptide of sequence SEQ ID NO: 1 corresponds to the Omomyc protein sequence. The term “Omomyc”, as used herein, refers to a polypeptide which consists of a mutated version of the bHLHZip domain of the Myc carrying the E61T, E681, R74Q and R75N mutations (wherein the numbering of the mutated positions is given with respect to the sequence of Myc region corresponding to amino acids 365-454 of the polypeptide as defined under accession number NP_002458 in the NCBI database, release of Mar. 15, 2015). The sequence of c-Myc provided in the NCBI database under the accession number NP_002458 is shown below (SEQ ID NO: 2), wherein the region from which Omomyc derives is shown underlined:

| (SEQ ID NO: 2) | |
|---|---|
| 1 | MDFFRVVENQ QPPATMPLNV SFTNRNYDLD YDSVQPYFYC DEEENFYQQQ QQSELQPPAP |
| 61 | SEDIWKKFEL LPTPPLSPSR RSGLCSPSYV AVTPFSLRGD NDGGGGSFST ADQLEMVTEL |
| 121 | LGGDMVNQSF ICDPDDETFI KNIIIQDCMW SGFSAAAKLV SEKLASYQAA RKDSGSPNPA |
| 181 | RGHSVCSTSS LYLQDLSAAA SECIDPSVVF PYPLNDSSSP KSCASQDSSA FSPSSDSLLS |
| 241 | STESSPQGSP EPLVLHEETP PTTSSDSEEE QEDEEEIDVV SVEKRQAPGK RSESGSPSAG |
| 301 | GHSKPPHSPL VLKRCHVSTH QHNYAAPPST RKDYPAAKRV KLDSVRVLRQ ISNNRKCTSP |
| 361 | RSSDTEENVK RRTHNVLERQ RRNELKRSFF ALRDQIPELE NNEKAPKVVI LKKATAYILS |
| 421 | VQAEEQKLIS EEDLLRKRRE QLKHKLEQLR NSCA |

Omomyc also contains the M2 domain of c-Myc, having the sequence RQRRNELKRSF (SEQ ID NO: 3) (see Dang and Lee, Mol. Cell. Biol., 1988, 8:4048-4054) (double underlined above), and which corresponds to a nuclear localization signal.

Omomyc is characterized in that it shows increased dimerization capacity with all three oncogenic Myc proteins (c-Myc, N-Myc and L-Myc). Omomyc can derive from the bHLHZip domain of any Myc protein known in the art, provided that the mutations which result in the tumor suppressor effect are preserved. Thus, the Omomyc that can be used in the present invention may derive from any mammal species, including but not being limited to domestic and farm animals (cows, horses, pigs, sheep, goats, dog, cats or rodents), primates and humans. Preferably, the Omomyc protein is derived from human Myc protein (accession number NP_002458, release of Mar. 12, 2019).

The term “Myc”, as used, herein, refers to a family of transcription factors which includes c-Myc, N-Myc and L-Myc. Myc protein activates expression of many genes through binding on consensus sequence CACGTG (Enhancer Box sequences or E-boxes and recruiting histone acetyl-transferases or HATs). However, Myc can also act as a transcriptional repressor. By binding the Miz-1 transcription factor and displacing p300 co-activator, it inhibits expression of Miz-1 target genes. Myc also has a direct role in the control of DNA replication.

The Myc b-HLH-LZ or Myc basic region helix-loop-helix leucine zipper domain refers to a region which determines Myc dimerization with Max protein and binding to Myc-target genes. This region corresponds to amino acids 365-454 of human Myc and is characterized by two alpha helices connected by a loop (Nair, S. K., & Burley, S. K., 2003, Cell, 112: 193-205).

In a preferred embodiment, the polypeptide of the invention is a polypeptide that comprises, consists of or consists essentially of the SEQ ID NO: 4 shown below.

(SEQ ID NO: 4)
MTEENVKRRTHNVLERQRRNELKRSFFALRDQIPELENNEKAPKVVILK
KATAYILSVQAETQKLISEIDLLRKQNEQLKHKLEQLRNSCA

In this context, “consisting essentially of” means that the specified molecule would not contain any additional sequences that would alter the activity of SEQ ID NO: 4.

Preferably, the polypeptide consists of SEQ ID NO: 4.

The term “functionally equivalent variant”, refers to any polypeptide which results from the insertion or addition of one or more amino acids and/or from the deletion of one or more amino acids and/or from the conservative substitution of one or more amino acids with respect to the polypeptide of SEQ ID NO: 1 and/or which results from the chemical modification of the polypeptide of SEQ ID NO: 1 and which substantially preserves the tumor suppressor activity of the SEQ ID NO: 1. Preferably, the functionally equivalent variant refers to any polypeptide which results from the insertion or addition of one or more amino acids and/or from the deletion of one or more amino acids and/or from the conservative substitution of one or more amino acids with respect to the polypeptide of SEQ ID NO: 1 and which substantially preserves the tumor suppressor activity of SEQ ID NO: 1; more preferably results from the insertion or addition of one or more amino acids with respect to the polypeptide of SEQ ID NO: 1.

The skilled person will understand that the preservation of the tumor suppressor activity requires that the variant can dimerize with Myc and/or its obligate partner p21/p22Max and inhibit Myc activity, that it is capable of translocating across the cell membrane and that it is capable of translocating across the nuclear envelope. In some embodiments, the functionally equivalent variant of the polypeptide of the invention homodimerizes less than Omomyc, or is not forced into homodimers by the formation of disulphide bridge. In particular the disulphide bridge formation in the homodimer form of certain embodiments of the polypeptide of the invention is less than in the polypeptide Omomyc.

“Less homodimerization”, as used herein, relates to the lower ability of forming obligate homodimers of the polypeptide of the invention even in reducing conditions. In a preferred embodiment, the ability is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% less than the ability of forming homodimers of Omomyc.

Reducing conditions, as used herein, relates to the presence of a reducing agent, a compound that donates an electron to another chemical species in a redox chemical reaction. Illustrative, non-limitative examples of reducing agents are DTT (dithiothreitol), b-mercaptoethanol or TCEP (tris(2-carboxyethyl)phosphine). It is possible that the amount of homodimers is the same in vitro, and that the difference between the functionally equivalent variant and Omomyc is present only in cells in presence of heterodimerization partners where the absence of the disulfide enables a potentially higher formation of heterodimers.

Several assays may be used to determine the homodimerization of a peptide, by way of illustrative non-limitative example by thermal denaturation monitored by circular dichroism, so dimerization may be detected through folding and thermal stability quantification.

Suitable functionally equivalent variants include polypeptides consisting essentially of the polypeptide of SEQ ID NO: 1. In this context, “consisting essentially of” means that the specified molecule would not contain any additional sequences that would alter the activity of the SEQ ID NO: 1.

In a preferred embodiment, the functionally equivalent variant of SEQ ID NO: 1 is a polypeptide which results from the insertion or addition of one or more amino acids with respect to the polypeptide of SEQ ID NO: 1. In an embodiment, the functionally equivalent variant results from the insertion of less than 10 amino acids, more preferably less than 5 amino acids, more preferably results from the insertion of one amino acid. In a preferred embodiment, results from the insertion of one amino acid that is methionine.

In another embodiment, the functionally equivalent variant of SEQ ID NO: 1 is a polypeptide which results from the deletion of one or more amino acids with respect to the polypeptide of SEQ ID NO: 1. In an embodiment, the functionally equivalent variant results from the deletion of less than 10 amino acids, more preferably less than 5 amino acids, more preferably results from the deletion of one amino acid.

Suitable functional variants of the targeting peptide are those showing a degree of identity with respect to the peptide of SEQ ID NO:1 of about greater than 25% amino acid sequence identity, such as 25%, 30%, 40%, 50%, 60%, 65%, 70%, 75%, 80%, 85%, 90%, 91%, 92%, 93%, 94%, 95%, 96%, 97%, 98% or 99%. The degree of identity between two polypeptides is determined using computer algorithms and methods that are widely known for the persons skilled in the art. The identity between two amino acid sequences is preferably determined by using the BLASTP algorithm as described previously (BLAST Manual, Altschul, S., et al., NCBI NLM NIH Bethesda, Md. 20894, Altschul, S., et al., J. Mol. Biol. 1990; 215: 403-410). In a preferred embodiment, the sequence identity is determined throughout the whole length of the polypeptide of SEQ ID NO: 1 or throughout the whole length of the variant or of both.

The functionally equivalent variants of the polypeptide of the invention may also include post-translational modifications, such as glycosylation, acetylation, isoprenylation, myristoylation, proteolytic processing, etc.

In another embodiment, suitable functional variants of the targeting peptide are those wherein one or more positions within the polypeptide of the invention contain an amino acid which is a conservative substitution of the amino acid present in the protein mentioned above. “Conservative amino acid substitutions” result from replacing one amino acid with another having similar structural and/or chemical properties. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Selection of such conservative amino acid substitutions is within the skill of one of ordinary skill in the art and is described, for example, by Dordo et al., (J. Mol. Biol, 1999, 217; 721-739) and Taylor et al., (J. Theor. Biol., 1986, 119:205-218).

It will be understood that in a preferred embodiment the functionally equivalent variants of Omomyc contain mutations at positions corresponding to the mutations E61T, E681, R74Q and R75N found in Omomyc derived from human c-Myc. The position wherein said mutations have to occur in the functionally equivalent variant can be determined by a multiple sequence alignment of different Myc sequences and identified by the alignment of those positions corresponding to positions 61, 68, 74 and 75 within the sequence of Omomyc derived from human c-Myc. In an embodiment, the functionally equivalent variants of Omomyc contain mutations at positions corresponding to the mutations E61T, E681, R74Q and R75N found in Omomyc derived from human c-Myc.

In another embodiment, the functionally equivalent variants of Omomyc contain mutations at positions corresponding to E61, E68, R74 and R75 within the sequence of Omomyc wherein E61 has been mutated to E61A or E61S; E68 has been mutated to E68L, E68M or E68V; R74 has been mutated to R74N; and R75 has been mutated to R75Q.

A multiple sequence alignment is an extension of pairwise alignment to incorporate more than two sequences at a time. Multiple alignment methods align all of the sequences in a given query set. A preferred multiple sequence alignment program (and its algorithm) is ClustalW, Clusal2W or ClustalW XXL (see Thompson et al. (1994) Nucleic Acids Res 22:4673-4680). Once the sequences of c-Myc from different organisms and of the variant are compared (aligned) as described herein, the skilled artisan can readily identify the positions within each of the sequence corresponding to positions E61T, E681, R74Q and R75N found in Omomyc and introduce within the Omomyc variant mutations corresponding to the E61T, E681, R74Q and R75N mutations found in Omomyc derived from human c-Myc.

Suitable assays for determining whether a polypeptide can be considered as a functionally equivalent variant of Omomyc include, without limitation:
Assays which measure the capacity of the polypeptide to form dimeric complexes with Max and Myc, such as the assays based on the expression of a reporter gene as described in Soucek et al. (Oncogene, 1998, 17: 2463-2472) as well as PLA (protein Ligation assay) or co-immunoprecipitation.
Assays which measure the capacity of the polypeptide to bind to the Myc/Max recognition site within DNA (the CACGTG site), such as the electrophoretic mobility shift assay (EMSA) described in Soucek et al. (supra.)
Assays which measure the capacity to repress Myc-induced transactivation, such as the assay based on the expression of a reporter gene under the control of the DNA binding sites specific for Myc/Max as described by Soucek et al. (supra.).
Assays based on the capacity of the polypeptide to inhibit growth of cells expressing the myc oncogene, as described by Soucek et al. (supra.).
Assays which measure the ability of the polypeptide to enhance myc-induced apoptosis, such as the assays described by Soucek et al. (Oncogene, 1998: 17, 2463-2472). Moreover, any assay commonly known in the art for assessing apoptosis in a cell can be used, such as the Hoechst staining, Propidium Iodide (PI) or Annexin V staining, trypan blue, DNA laddering/fragmentation and TUNEL.

In a preferred embodiment, a polypeptide is considered a functionally equivalent variant of Omomyc if it shows an activity in one or more of the above assays which is at least 10%, 20%, 30%, 40%, 50%, 60%, 70%, 80%, 90% or 100% of the native Omomyc.

In a particular embodiment, the functionally equivalent variant of the polypeptide of SEQ ID NO: 1 comprises the polypeptide of SEQ ID NO: 1, wherein the residue X at position 89 of SEQ ID NO: 1 is not a cysteine. Preferably, the residue X at position 89 of SEQ ID NO: 1 is an aliphatic amino acid, or a sulfured amino acid, or a dicarboxylic amino acid or their amides, or an amino acid having two basic groups, or an aromatic amino acid, or a cyclic amino acid, or a hydroxylated amino acid. More preferably is an amino acid selected from serine, threonine and alanine, preferably selected from serine and alanine.

Suitable functionally equivalent variants of SEQ ID NO: 1 having a residue X at position 89 of SEQ ID NO: 1 which is not a cysteine are disclosed in the following table.

| SEQ ID NO | SEQUENCE |
|---|---|
| SEQ ID NO: | TEENVKRRTHNVLERQRRNELKRSFFALRDQIPELENNEKAPKVVILKKATAYILSV |
| 5 | QAETQKLISEIDLLRKQNEQLKHKLEQLRNSXA (wherein X is any aa different |
| | from Cys) |
| SEQ ID NO: | MTEENVKRRTHNVLERQRRNELKRSFFALRDQIPELENNEKAPKVVILKKATAYILS |
| 6 | VQAETQKLISEIDLLRKQNEQLKHKLEQLRNSXA (wherein X is any aa different |
| | from Cys) |
| SEQ ID NO: | TEENVKRRTHNVLERQRRNELKRSFFALRDQIPELENNEKAPKVVILKKATAYILSV |
| 7 | QAETQKLISEIDLLRKQNEQLKHKLEQLRNSSA |
| SEQ ID NO: | MTEENVKRRTHNVLERQRRNELKRSFFALRDQIPELENNEKAPKVVILKKATAYILS |
| 8 | VQAETQKLISEIDLLRKQNEQLKHKLEQLRNSSA |
| SEQ ID NO: | TEENVKRRTHNVLERQRRNELKRSFFALRDQIPELENNEKAPKVVILKKATAYILSV |
| 9 | QAETQKLISEIDLLRKQNEQLKHKLEQLRNSAA |
| SEQ ID NO: | MTEENVKRRTHNVLERQRRNELKRSFFALRDQIPELENNEKAPKVVILKKATAYILS |
| 10 | VQAETQKLISEIDLLRKQNEQLKHKLEQLRNSAA |

Thus, in a preferred embodiment, the functionally equivalent variant of the polypeptide of SEQ ID NO: 1 is selected from the group consisting of SEQ ID NO: 4, SEQ ID NO: 5, SEQ ID NO: 6, SEQ ID NO: 7, SEQ ID NO: 8, SEQ ID NO: 9 and SEQ ID NO: 10. Preferably, the functionally equivalent variant is SEQ ID NO: 4.

Additionally, functionally equivalent variants of Omomyc are also capable of transducing cells after the variant is contacted with said cell. It will be understood that functionally equivalent variants of Omomyc contain the protein transducing domain found in native Omomyc or another functional protein transducing domain.

In a preferred embodiment, a polypeptide is considered as a functionally equivalent variant of SEQ ID NO: 1 if it is capable of transducing a target cell at least 10%, 20%, 30%, 40%, 50%, 60%, 70%, 80%, 90% or 100% as efficiently as SEQ ID NO: 1.

Additionally, functionally equivalent variants of SEQ ID NO: 1 are also capable of translocating to the nucleus of the target tumor cell.

In a preferred embodiment, a polypeptide is considered as a functionally equivalent variant of SEQ ID NO: 1 if it is capable of translocating to the nucleus of the target tumor cells at least 10%, 20%, 30%, 40%, 50%, 60%, 70%, 80%, 90% or 100% as efficiently as the SEQ ID NO: 1.

Suitable assays for determining whether a polypeptide is a functionally equivalent variant of SEQ ID NO: 1 in terms of its ability to translocate across the cellular membrane and to the nucleus include double labelling of a cell with a reagent specific for the polypeptide and with a dye which specifically labels the nucleus of the cell (such as DAPI or Hoechst dye).

The detection of the polypeptide of the invention can be performed by confocal microscopy or by fluorescence microscopy.

In another preferred embodiment, compound (i) of the invention is a conjugate comprising a polypeptide comprising the sequence SEQ ID NO: 1 or a functionally equivalent variant thereof and a chemical moiety that facilitates cellular uptake of the polypeptide or of the functionally equivalent variant thereof.

The term “conjugate”, as used herein, refers to two or more compounds which are covalently linked together so that the function of each compound is retained in the conjugate.

The term “chemical moiety” refers to any chemical compound containing at least one carbon atom. Examples of chemical moieties include, but are not limited to, any peptide chain enriched in hydrophobic amino acids and hydrophobic chemical moieties.

In preferred embodiments, the conjugate according to the invention comprises at least 1, at least 2, at least 3, at least 4, at least 5, at least 6, at least 7, at least 8, at least 9, at least 10 or more chemical moieties that facilitate cellular uptake of the polypeptide or of the functionally equivalent variant of said polypeptide.

In one embodiment, the chemical moiety that facilitates cellular uptake of the polypeptide is a lipid or a fatty acid.

A fatty acid generally is a molecule comprising a carbon chain with an acidic moiety (e.g., carboxylic acid) at an end of the chain. The carbon chain of a fatty acid may be of any length, however, it is preferred that the length of the carbon chain be of at least 2, 3, 4, 5, 6, 7, 8, 9, 10, 11, 12, 13, 14, 15, 16, 17, 18, 19, 20 or more carbon atoms, and any range derivable therein. In certain embodiments, the length of the carbon chain is from 4 to 18 carbon atoms in the chain portion of the fatty acid. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated. The fatty acid may be branched, though in preferable embodiments of the present invention, it is unbranched. Specific fatty acids include, but are not limited to, linoleic acid, oleic acid, palmitic acid, linolenic acid, stearic acid, lauric acid, myristic acid, arachidic acid, palmitoleic acid, and arachidonic acid.

In a preferred embodiment, the chemical moiety that facilitates the cellular uptake of the polypeptide comprising the sequence SEQ ID NO: 1 or a functionally equivalent variant thereof, is a cell penetrating peptide sequence, in which case, the conjugate would comprise a fusion protein comprising the polypeptide comprising SEQ ID NO: 1 or the functionally equivalent variant thereof and the cell penetrating peptide sequence.

The term “fusion protein” relates to proteins generated by gene technology which consist of two or more functional domains derived from different proteins. A fusion protein may be obtained by conventional means, e.g., by means of gene expression of the nucleotide sequence encoding for said fusion protein in a suitable cell. It will be understood that the cell penetrating peptide refers to a cell penetrating peptide which is different from the cell penetrating peptide which forms part of the polypeptide comprising SEQ ID NO: 1 or of the functionally equivalent variant of SEQ ID NO: 1.

The term “cell penetrating peptide sequence” is used in the present specification interchangeably with “CPP”, “protein transducing domain” or “PTD”. It refers to a peptide chain of variable length that directs the transport of a protein inside a cell. The delivering process into cell commonly occurs by endocytosis but the peptide can also be internalized into cell by means of direct membrane translocation. CPPs typically have an amino acid composition that either contains a high relative abundance of positively charged amino acids such as lysine or arginine or has sequences that contain an alternating pattern of polar/charged amino acid and non-polar, hydrophobic amino acids.

Examples of CPPs which can be used in the present invention include, without limitation, the CPP found in Drosophila antennapedia protein (RQIKIWFQNRRMKWKK. SEQ ID NO:13), the CPP found in the herpesvirus simplex 1 (HSV-1) VP22 DNA-binding protein (DAATATRGRSAASRPTERPRAPARSASRPRRPVE, SEQ ID NO:14), the CPP of Bac-7 (RRIRPRPPRLPRPRPRPLPFPRPG; SEQ ID NO: 15), the CPPs of the HIV-1 TAT protein consisting of amino acids 49-57 (RKKRRQRRR, SEQ ID NO: 16), amino acids 48-60 (GRKKRRQRRRTPQ, SEQ ID NO: 17), amino acids 47-57 (YGRKKRRQRRR; SEQ ID NO: 18); the CPP of S413-PV peptide (ALWKTLLKKVLKAPKKKRKV; SEQ ID NO: 19), the CPP of penetratin (RQIKWFQNRRMKWKK; SEQ ID NO: 20), the CPP of SynB1 (RGGRLSYSRRRFSTSTGR; SEQ ID NO: 21), the CPP of SynB3 (RRLSYSRRRF; SEQ ID NO: 22), the CPP of PTD-4 (PIRRRKKLRRLK; SEQ ID NO: 23), the CPP of PTD-5 (RRQRRTSKLMKR; SEQ ID NO: 24), the CPP of the FHV Coat-(35-49) (RRRRNRTRRNRRRVR; SEQ ID NO: 25), the CPP of BMV Gag-(7-25) (KMTRAQRRAAARRNRWTAR; SEQ ID NO: 26), the CPP of HTLV-II Rex-(4-16) (TRRQRTRRARRNR; SEQ ID NO: 27), the CPP of D-Tat (GRKKRRQRRRPPQ; SEQ ID NO:28), the CPP R9-Tat (GRRRRRRRRRPPQ; SEQ ID NO: 29), the CPP of MAP (KLALKLALKLALALKLA; SEQ ID NO: 30), the CPP of SBP (MGLGLHLLVLAAALQGAWSQPKKKRKV; SEQ ID NO: 31), the CPP of FBP (GALFLGWLGAAGSTMGAWSQPKKKRKV; SEQ ID NO: 32), the CPP of MPG (ac-GALFLGFLGAAGSTMGAWSQPKKKRKV-cya; SEQ ID NO: 33), the CPP of MPG(ENLS) (ac-GALFLGFLGAAGSTMGAWSQPKSKRKV-cya; SEQ ID NO: 34), the CPP of Pep-1 (ac-KETWWETWWTEWSQPKKKRKV-cya; SEQ ID NO: 35), the CPP of Pep-2 (ac-KETWFETWFTEWSQPKKKRKV-cya; SEQ ID NO: 36), a polyarginine sequence having the structure RN (wherein N is between 4 and 17), the GRKKRRQRRR sequence (SEQ ID NO: 37), the RRRRRRLR sequence (SEQ ID NO: 38), the RRQRRTS KLMKR sequence (SEQ ID NO: 39); Transportan GWTLNSAGYLLGKINLKALAALAKKIL (SEQ ID NO: 40); KALAWEAKLAKALAKALAKHLAKALAKALKCEA (SEQ ID NO: 41); RQIKIWFQNRRMKWKK (SEQ ID NO: 42), the YGRKKRRQRRR sequence (SEQ ID NO: 43); the RKKRRQRR sequence (SEQ ID NO: 44); the YARAAARQARA sequence (SEQ ID NO: 45); the THRLPRRRRRR sequence (SEQ ID NO: 46); the GGRRARRRRRR sequence (SEQ ID NO: 47).

In a preferred embodiment, said cell-penetrating peptide is not the endogenous contained in SEQ ID NO: 1.

In a preferred embodiment, the CPP is the CPP of the HIV-1 TAT protein consisting of amino acids 49-57 (RKKRRQRRR, SEQ ID NO: 16). In another preferred embodiment the CPP is the GRKKRRQRRR sequence (SEQ ID NO: 37) or RRRRRRLR (SEQ ID NO: 38).

In another embodiment, the CPP is the GRKKRRQRRR sequence (SEQ ID NO: 37) or RRRRRRRR (SEQ ID NO: 65).

In some embodiments, a CPP is as a CPP as described in WO2019/018898, the content of which is incorporated herein by reference in its entirety.

In one embodiment, the cell-penetrating peptide sequence is fused at the N-terminus of the polypeptide of the invention or of the functionally equivalent variant of said polypeptide. In another embodiment, the cell-penetrating peptide is fused at the C-terminus of the polypeptide of the invention or of the functionally equivalent variant of said polypeptide.

In preferred embodiments, the conjugates or fusion proteins of the combination according to the invention comprise, in addition to the own cell penetrating peptide found in the polypeptide of SEQ ID NO: 1 or of the functionally equivalent variant of said polypeptide, at least 1, at least 2, at least 3, at least 4, at least 5, at least 6, at least 7, at least 8, at least 9, at least 10 or more additional cell penetrating peptides.

Suitable fusion proteins of the invention include the polypeptides Omomyc*TAT and Omomyc*LZArg as defined below:

| Name | SEQ ID NO: | Sequence |
|---|---|---|
| Omomyc*TAT | 11 | MTEENVKRRTHNVLERQRRNELKRSFFALRDQIPELENNEKAPKVVI |
| | | LKKATAYILSVQAETQKLISEIDLLRKQNEQLKHKLEQLRNSCAGRK |
| | | KRRQRRR |
| Omomyc*LZArg | 12 | MTEENVKRRTHNVLERQRRNELKRSFFALRDQIPELENNEKAPKVVI |
| | | LKKATAYILSVQAETQKLISEIDLLRKQNEQLKHKLEQLRNSCARRR |
| | | RRRLR |

Thus, in a preferred embodiment the fusion protein is the polypeptide selected from SEQ ID NO: 11 and 12.

Suitable assays for determining whether a conjugate preserves the cell membrane translocation capacity of Omomyc include, without limitation, assays which measure the capacity of the conjugate to transduce cells in culture. This assay is based on contacting the conjugate with culture cells and detecting the presence of the conjugate in an intracellular location.

In another preferred embodiment, the conjugate of the combination of the invention additionally comprises a further nuclear localization signal.

The term “nuclear localization signal” (NLS), as used herein, refers to an amino acid sequence of about 4-20 amino acid residues in length, which serves to direct a protein to the nucleus. Typically, the nuclear localization sequence is rich in basic amino acids and exemplary sequences are well known in the art (Gorlich D. (1998) EMBO 5.17:2721-7). In some embodiments, the NLS is selected from the group consisting of the SV40 large T Antigen NLS (PKKKRKV, SEQ ID NO: 48); the Nucleoplasmin NLS (KRPAATKKAGQAKKKK, SEQ ID NO: 49); the CBP80 NLS (RRRHSDENDGGQPHKRRK, SEQ ID NO: 50); the HIV-1 Rev protein NLS (RQARRNRRRWE, SEQ ID NO: 51); the HTLV-I Rex (MPKTRRRPRRSQRKRPPT, SEQ ID NO: 52); the hnRNP A NLS (NQSSNFGPMKGGNFGGRSSGPYGGGGQYFKPRNQGGY, SEQ ID NO: 53); the rpL23a NLS (VHSHKKKKIRTSPTFTTPKTLRLRRQPKYPRKSAPRRNKLDHY, SEQ ID NO: 54). In one embodiment of the invention, the nuclear localization signal comprises the motif K (K/R)×(K/R).

In an even more preferred embodiment, the nuclear localization signal is selected from the group consisting of PKKKRKV (SEQ ID NO: 48), PAAKRVKLD (SEQ ID NO: 56) and KRPAATKKAGQ AKKKK (SEQ ID NO: 49).

In another preferred embodiment, the NLS may be N-terminal or C-terminal to the conjugate or the fusion protein comprising the polypeptide of SEQ ID NO: 1 or a functionally equivalent variant thereof.

The skilled person will understand that it may be desirable that the conjugate of the invention further comprises one or more flexible peptides that connect the polypeptide comprising SEQ ID NO: 1 or the functionally equivalent variant thereof, the cell penetrating peptide sequence and/or the NLS. Thus, in a particular embodiment the polypeptide comprising SEQ ID NO: 1 or a functionally equivalent variant thereof is directly connected to the cell penetrating peptide sequence. In another particular embodiment, the polypeptide comprising SEQ ID NO: 1 or a functionally equivalent variant thereof is connected to the cell penetrating peptide sequence through a flexible peptide. In an embodiment the polypeptide comprising SEQ ID NO: 1 or the functionally variant thereof is directly connected to the NLS. In another embodiment the polypeptide comprising SEQ ID NO: 1 or a functionally equivalent variant thereof is connected to the NLS through a flexible peptide.

In a particular embodiment the polypeptide of the conjugate according to the invention is directly connected to the cell penetrating peptide sequence and to the NLS.

In one embodiment, the NLS is one of the NLS which appears endogenously in the Myc sequence, such as the M1 peptide (PAAKRVKLD, SEQ ID NO: 56) or the M2 peptide (RQRRNELKRSF, SEQ ID NO: 57).

In another embodiment the additional NLS refers to an NLS which is different to the endogenous NLS found in a polypeptide comprising SEQ ID NO: 1 or in the functionally equivalent variant of SEQ ID NO: 1.

In preferred embodiments, the conjugates or fusion proteins according to the invention comprise, in addition to the endogenous NLS found in the polypeptide of the invention or in the functionally equivalent variant thereof, at least 1, at least 2, at least 3, at least 4, at least 5, at least 6, at least 7, at least 8, at least 9, at least 10 NLS.

In another particular embodiment, the polypeptide of the conjugate fore use according to the invention is connected to the cell penetrating peptide sequence through a first flexible peptide linker and to the NLS through a second flexible peptide linker.

As used herein, the term “flexible peptide”, “spacer peptide” or “linker peptide” refers to a peptide that covalently binds two proteins or moieties but which is not part of either polypeptide, allowing movement of one with respect to the other, without causing a substantial detrimental effect on the function of either the protein or the moiety. Thus, the flexible linker does not affect the tumour tracing activity of the polypeptide sequence, the cell penetrating activity of the cell penetrating peptide or the nuclear localization capacity of the NLS.

The flexible peptide comprises at least one amino acid, at least two amino acids, at least three amino acids, at least four amino acids, at least five amino acids, at least six amino acids, at least seven amino acids, at least eight amino acids, at least nine amino acids, at least 10 amino acids, at least 12 amino acids, at least 14 amino acids, at least 16 amino acids, at least 18 amino acids, at least 20 amino acids, at least 25 amino acids, at least 30 amino acids, at least 35 amino acids, at least 40 amino acids, at least 45 amino acids, at least 50 amino acids, at least 60 amino acids, at least 70 amino acids, at least 80 amino acids, at least 90 amino acids, or about 100 amino acids. In some embodiments the flexible peptide will permit the movement of one protein with respect to the other in order to increase solubility of the protein and/or to improve its activity. Suitable linker regions include a poly-glycine region, the GPRRRR sequence (SEQ ID NO: 58) of combinations of glycine, proline and alanine residues.

In a particular embodiment, the conjugates according to the invention comprise a tag bound to the conjugate or to the C-terminal or N-terminal domain of said polypeptide or fusion protein or variant thereof. Said tag is generally a peptide or amino acid sequence which can be used in the isolation or purification of said fusion protein. Thus, said tag is capable of binding to one or more ligands, for example, one or more ligands of an affinity matrix such as a chromatography support or bead with high affinity. An example of said tag is a histidine tag (His-tag or HT), such as a tag comprising 6 residues of histidine (His6 or H6), which can bind to a column of nickel (Ni²⁺) or cobalt (Co²⁺) with high affinity. His-tag has the desirable feature that it can bind its ligands under conditions that are denaturing to most proteins and disruptive to most protein-protein interactions. Thus, it can be used to remove the bait protein tagged with H6 following the disruption of protein-protein interactions with which the bait has participated.

Additional illustrative, non-limitative, examples of tags useful for isolating or purifying the conjugate or the polypeptide comprising SEQ ID NO: 1 or a variant thereof or a fusion protein include Arg-tag, FLAG-tag (DYKDDDDK; SEQ ID NO:59), Strep-tag (WSHPQFEK, SEQ ID NO:60), an epitope capable of being recognized by an antibody, such as c-myc-tag (recognized by an anti-c-myc antibody), HA tag (YPYDVPDYA, SEQ ID NO:61), V5 tag (GKPIPNPLLGLDST, SEQ ID NO:62), SBP-tag, S-tag, calmodulin binding peptide, cellulose binding domain, chitin binding domain, glutathione S-transferase-tag, maltose binding protein, NusA, TrxA, DsbA, Avi-tag, etc. (Terpe K., Appl. Microbiol. Biotechnol. 2003, 60:523-525), an amino acid sequence such as AHGHRP (SEQ ID NO:63) or PIHDHDHPHLVIHSGMTCXXC (SEQ ID NO:64), β-galactosidase and the like.

The tag can be used, if desired, for the isolation or purification of said fusion protein.

In another preferred embodiment, compound (i) of the invention is a polynucleotide encoding the polypeptide or the fusion protein disclosed above. In a preferred embodiment, compound (i) of the invention is a polynucleotide encoding a polypeptide comprising the sequence SEQ ID NO: 1 or a functionally equivalent variant thereof. In another embodiment, compound (i) of the invention is a polynucleotide encoding a conjugate comprising the polypeptide comprising the sequence SEQ ID NO: 1 or a functionally equivalent variant thereof and a chemical moiety that facilitates cellular uptake of the polypeptide or of the functionally equivalent variant thereof; more preferably is a polynucleotide encoding a fusion protein between the polypeptide comprising the sequence SEQ ID NO: 1 or a functionally equivalent variant thereof and a cell-penetrating peptide sequence.

The terms “polynucleotide”, “nucleic acid” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In addition to a native nucleic acid molecule, a nucleic acid molecule of the present invention may also comprise modified nucleic acid molecules. As used herein, mRNA refers to an RNA that can be translated in a cell.

In preferred embodiment, the polynucleotide of the invention is an mRNA. mRNA can be chemically synthesized, can be obtained by means of in vitro transcription or can be synthesized in vivo in the target cell. The nucleotide sequences that form the polynucleotide encoding the conjugate or fusion protein of the invention are in the same correct reading frame for expression thereof.

In a preferred embodiment, component (i) of the combination of the invention is an mRNA encoding for a polypeptide consisting of the sequence SEQ ID NO: 1 or a polypeptide consisting of a functionally equivalent variant of SEQ ID NO: 1 or a polypeptide consisting of SEQ ID NO: 4.

In another embodiment, component (i) of the combination of the invention is a vector comprising a polynucleotide of the invention.

The term “vector”, as used herein, refers to a nucleic acid sequence comprising the necessary sequences so that after transcribing and translating said sequences in a cell a polypeptide encoded by the polynucleotide of the invention is generated. Said sequence is operably linked to additional segments that provide for its autonomous replication in a host cell of interest. Preferably, the vector is an expression vector, which is defined as a vector which, in addition to the regions of the autonomous replication in a host cell, contains regions operably linked to the nucleic acid of the invention and which are capable of enhancing the expression of the products of the nucleic acid according to the invention. The vectors of the invention can be obtained by means of techniques widely known in the art. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. Suitable vectors comprising a polynucleotide of the invention are vectors derived from expression vectors in prokaryotes such as pUC18, pUC19, pBluescript and their derivatives, mp18, mp19, pBR322, pMB9, CoIE1, pCRI, RP4, phages and “shuttle” vectors such as pSA3 and pAT28, expression vectors in yeasts such as vectors of the 2-micron plasmid type, integration plasmids, YEP vectors, centromeric plasmids and similar, expression vectors in insect cells such as the vectors of the pAC series and of the pVL series, expression vectors in plants such as vectors of the series plBI, pEarleyGate, pAVA, pCAMBIA, pGSA, pGWB, pMDC, pMY, pORE and similar and expression vectors in superior eukaryote cells based on viral vectors (adenovirus, virus associated to adenovirus as well as retrovirus and, in particular, lentivirus) as well as non-viral vectors such as pSilencer 4.1-CMV (Ambion), pcDNA3, pcDNA3.1/hyg, pHCMV/Zeo, pCR3.1, pEFI/His, pIND/GS, pRc/HCMV2, pSV40/Zeo2, pTRACER-HCMV, pUB6/V5-His, pVAXI, pZeoSV2, pCl, pSVL, pKSV-10, pBPV-1, pML2d and pTDT1. In a preferred embodiment, the polynucleotide of the invention is comprised in a vector selected from the group consisting of pEGFP or pBabe retroviral vectors and pTRIPZ or pSLIK lentiviral vectors.

The vector of the invention may be used to transform, transfect or infect cells that can be transformed, transfected or infected by said vector. Said cells may be prokaryotic or eukaryotic.

The vector preferably comprises the polynucleotide of the invention operationally bound to sequences that regulate the expression of the polynucleotide of the invention. The regulatory sequences of use in the present invention may be nuclear promoters or, alternatively, enhancer sequences and/or other regulatory sequences that increase expression of the heterologous nucleic acid sequence. In principle, any promoter can be used in the present invention provided said promoter is compatible with the cells wherein the polynucleotide is to be expressed. Thus, promoters suitable for realizing the present invention include, but are not necessarily limited to, constitutive promoters such as derivatives of eukaryotic virus genomes such as polyoma virus, adenovirus, SV40, CMV, avian sarcoma virus, hepatitis B virus, the metallothionein gene promoter, the herpes simplex virus thymidine kinase gene promoter, LTR regions of retroviruses, the immunoglobulin gene promoter, the actin gene promoter, the EF-1alpha gene promoter as well as inducible promoters wherein protein expression depends on the addition of a molecule or exogenous signal, such as tetracycline systems, the NFκB/UV light system, the Cre/Lox system and the heat shock genes promoter, the regulable RNA polymerase II promoters described in WO/2006/135436 and tissue-specific promoters.

In another embodiment, component (i) of the combination of the invention is a cell capable of secreting into the medium the polypeptide of the invention or the conjugate of the invention, preferably the polypeptide of the invention or the fusion protein of the invention.

Suitable cells capable of secreting a polypeptide of the invention include without limitation, cardiomyocytes, adipocytes, endothelial cells, epithelial cells, lymphocytes (B and T cells), mastocytes, eosinophils, vascular intima cells, primary cultures of isolated cells of different organs, preferably of cells isolated from Langerhans islets, hepatocytes, leukocytes, including mononuclear leukocytes, mesenchymal, umbilical cord or adult (of skin, lung, kidney and liver), osteoclasts, chondrocytes and other connective tissue cells. Cells of established lines such as Jurkat T cells, NIH-3T3, CHO, Cos, VERO, BHK, HeLa, COS, MDCK, 293, 3T3 cells, C2C12 myoblasts and W138 cells are also suitable. Persons skilled in the art will appreciate that the cells capable of secreting into the medium a polypeptide of the invention may be found forming microparticles or microcapsules so that the cells have a greater useful life in patients. Materials suitable for the formation of microparticles object of the invention include any biocompatible polymeric material which permits continuous secretion of the therapeutic products and which acts as support of the cells. Thus, said biocompatible polymeric material may be, for example, thermoplastic polymers or hydrogen polymers. Among the thermoplastic polymers we have acrylic acid, acrylamide, 2-aminoethyl methacrylate, poly(tetrafluoroethylene-cohexafluorpropylene), methacrylic-(7-cumaroxy) ethyl ester acid, N-isopropyl acrylamide, polyacrylic acid, polyacrylamide, polyamidoamine, poly(amino)-p-xylylene, poly(chloroethylvinylether), polycaprolactone, poly(caprolactone-co-trimethylene carbonate), poly(carbonate urea) urethane, poly(carbonate) urethane, polyethylene, polyethylene and acrylamide copolymer, polyethylene glycol, polyethylene glycol methacrylate, poly(ethylene terephthalate), poly(4-hydroxybutyl acrylate), poly(hydroxyethyl methacrylate), poly(N-2-hydroxypropyl methacrylate), poly(lactic glycolic acid), poly(L-lactic acid), poly(gamma-methyl, Lglutamate), poly(methylmethacrylate), poly(propylene fumarate), poly(propylene oxide), polypyrrole, polystyrene, poly(tetrafluoroethylene), polyurethane, polyvinyl alcohol, polyethylene of ultra-high molecular weight, 6-(p-vinylbenzamide)-hexanoic acid N-pvinybenzyl-D-maltonamide and copolymers containing more than one of said polymers. Among the polymers of hydrogel type we have natural materials of alginate, agarose, collagen, starch, hyaluronic acid, bovine serum albumin, cellulose and their derivatives, pectin, chondroitin sulphate, fibrin and fibroin, as well as synthetic hydrogels such as Sepharose® and Sephadex®.

### Compound (ii) of the Combination of the Invention

Compound (ii) of the combination of the invention is a MEK inhibitor.

“MEK” (E.C. 2.7.12.2), as used herein, refers to mitogen-activated protein kinase kinase, also known as dual specificity mitogen-activated protein kinase kinase, MAP kinase kinase, MAPKK, MAP2K, MKK, ERK activator kinase or MAPK/ERK kinase. MEK refers to the family of mitogen/extracellular signal-regulated kinases that are kinase enzymes which phosphorylate mitogen-activated protein kinase (MAPK). Isoforms of MEK include, but are not limited to, MEK1 and MEK2. Therefore, in a preferred embodiment, MEK is selected from the group consisting of MEK1 and/or MEK2.

As used herein, the term “MEK1” means mitogen-activated protein kinase kinase 1. The sequence of MEK1 protein in humans corresponds to the sequence Q02750 in the UniProt Database (version 234 of the entry as of 3 May 2023). There are two isoforms (MKK1a and MKK1b) produced by alternative splicing. This protein is encoded by the MAP2K1 gene.

As used herein, the term “MEK2” means mitogen-activated protein kinase kinase 2. The sequence of MEK2 protein in humans corresponds to the sequence P36507 in the UniProt Database (version 239 of the entry as of 3 May 2023). This protein is encoded by the MAP2K2 gene.

Both MEK1 and MEK2 function specifically in the MAPK/ERK cascade, and catalyse the concomitant phosphorylation of a threonine and a tyrosine residue in a Thr-Glu-Tyr sequence located in the extracellular signal-regulated kinases MAPK3/ERK1 and MAPK1/ERK2, leading to their activation and further transduction of the signal within the MAPK/ERK cascade.

Unless otherwise indicated herein, use of the term “MEK” may refer to MEK1, MEK2, or a combination thereof.

“MEK inhibitor”, as used herein, relates to any compound capable of causing a decrease in the activity of MEK, particularly in the activity of MEK1 and/or MEK2, including those compounds which prevent expression of MEK gene, particularly MEK1 and/or MEK2 genes, and compounds which lead to reduced MEK mRNA or protein levels, particularly MEK1 and/or MEK2 mRNA or protein levels. MEK inhibitors primarily inhibit the catalytic activity of MEK1 and/or MEK2 enzymes. Therefore, in a preferred embodiment, the MEK inhibitor is selected from the group consisting of a MEK1 inhibitor, a MEK2 inhibitor and an inhibitor of both MEK1 and MEK2. A MEK inhibitor prevents activation of ERK1/2 by MEK1/2.

The expression of a protein or nucleic acid is considered reduced when its levels decrease with respect to the reference value by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100% (i.e., absent).

The reference value refers to the level of protein or nucleic acid in control subject, which may be a subject who does not suffer a specific disease.

Suitable methods for determining whether an inhibitor is capable of decreasing MEK mRNA levels, particularly MEK1 and/or MEK2 mRNA levels include, without limitation, standard assays for determining mRNA expression levels such as qPCR, RT-PCR, RNA protection analysis, Northern blot, RNA dot blot, in situ hybridization, microarray technology, tag based methods such as serial analysis of gene expression (SAGE) including variants such as LongSAGE and SuperSAGE, microarrays, fluorescence in situ hybridization (FISH), including variants such as Flow-FISH, qFiSH and double fusion FISH (D-FISH), and the like. Preferably quantitative or semi-quantitative RT-PCR is preferred. Real-time quantitative or semi-quantitative RT-PCR is particularly advantageous.

The nucleic acid contained in the sample (e.g., cell or tissue prepared from the subject) is first extracted according to standard methods, for example using lytic enzymes or chemical solutions or extracted by nucleic-acid-binding resins following the manufacturer's instructions.

In case the mRNA is measured in a biological sample, said biological sample may be treated to physically, mechanically or chemically disrupt tissue or cell structure, to release intracellular components into an aqueous or organic solution to prepare nucleic acids for further analysis. The nucleic acids are extracted from the sample by procedures known to the skilled person and commercially available. RNA is then extracted from frozen or fresh samples by any of the methods typical in the art, for example, Sambrook, J., et al., 2001. Molecular cloning: A Laboratory Manual, 3^{rd} ed., Cold Spring Harbor Laboratory Press, N.Y., Vol. 1-3. Preferably, care is taken to avoid degradation of the RNA during the extraction process.

The expression level can be determined using mRNA obtained from a formalin-fixed, paraffin-embedded tissue sample. mRNA may be isolated from an archival pathological sample or biopsy sample which is first deparaffinized. An exemplary deparaffinization method involves washing the paraffinized sample with an organic solvent, such as xylene. Deparaffinized samples can be rehydrated with an aqueous solution of a lower alcohol. Suitable lower alcohols, for example, include methanol, ethanol, propanols and butanols. Deparaffinized samples may be rehydrated with successive washes with lower alcoholic solutions of decreasing concentration, for example. Alternatively, the sample is simultaneously deparaffinized and rehydrated. The sample is then lysed and RNA is extracted from the sample. Samples can be also obtained from fresh tumor tissue such as a resected tumor. In a particular embodiment samples can be obtained from fresh tumor tissue or from OCT embedded frozen tissue.

In order to normalize the values of mRNA expression among the different samples, it is possible to compare the expression levels of the mRNA of interest in the test samples with the expression of a control RNA. A “control RNA”, as used herein, relates to RNA whose expression levels do not change or change only in limited amounts in tumor cells with respect to non-tumorigenic cells. Preferably, the control RNA is mRNA derived from housekeeping genes and which code for proteins which are constitutively expressed and carry out essential cellular functions. Preferred housekeeping genes for use in the present invention include β-2-microglobulin, ubiquitin, 18-S ribosomal protein, cyclophilin, IPO8, HPRT, GAPDH, PSMB4, tubulin and β-actin.

The relative gene expression quantification may be calculated according to the comparative threshold cycle (Ct) method using a housekeeping gene as an endogenous control and commercial RNA controls as calibrators. Final results are determined according to the formula 2^{-(ΔCtsample-ΔCt calibrator)} where ΔCt values of the calibrator and sample are determined by subtracting the Ct value of the target gene from the value of the control gene.

Suitable methods for determining whether an inhibitor acts by decreasing the MEK protein levels, particularly the MEK1 and/or MEK2 protein levels, include the quantification by means of conventional methods, for example, using antibodies with a capacity to specifically bind to the proteins encoded by MEK genes, particularly MEK1 and/or MEK2 genes (or to fragments thereof containing antigenic determinants) and subsequent quantification of the resulting antibody-antigen complexes.

The antibodies to be employed in these assays can be, for example, polyclonal sera, hybridoma supernatants or monoclonal antibodies, antibody fragments, Fv, Fab, Fab′ and F(ab′)2, ScFv, diabodies, triabodies, tetrabodies and humanized antibodies. At the same time, the antibodies can be labeled or not. Illustrative, but non-exclusive examples of markers which can be used include radioactive isotopes, enzymes, fluorophores, chemiluminescent reagents, enzymatic substrates or cofactors, enzymatic inhibitors, particles, colorants, etc. There are a wide variety of well-known assays that can be used in the present invention, which use non-labeled antibodies (primary antibody) and labeled antibodies (secondary antibodies); among these techniques are included Western blot or Western transfer, ELISA (enzyme linked immunosorbent assay), RIA (radioimmunoassay), competitive EIA (enzymatic immunoassay), DAS-ELISA (double antibody sandwich ELISA), immunocytochemical and immunohistochemical techniques, techniques based on the use of biochips or protein microarrays including specific antibodies or assays based on colloidal precipitation in formats such as dipsticks. Other ways of detecting and quantifying the levels of the protein of interest include techniques of affinity chromatography, binding-ligand assays, etc.

On the other hand, the determination of the levels of the MEK protein, particularly MEK1 and/or MEK2 protein, can be carried out by constructing a tissue microarray (TMA) containing the subject samples assembled, and determining the expression levels of the corresponding protein by immunohistochemistry techniques. Immunostaining intensity can be evaluated by two or more different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, and low expression (1+) versus moderate (2+) and high (3+) expression, taking into account the expression in tumor cells and the specific cut-off for each marker. As a general criterion, the cut-offs are selected in order to facilitate reproducibility, and when possible, to translate biological events. Alternatively, the immunostaining intensity can be evaluated by using imaging techniques and automated methods such as those disclosed in Rojo, M. G. et al. (Folia Histochem. Cytobiol. 2009; 47: 349-54) or Mulrane, L. et al. (Expert Rev. Mol. Diagn. 2008; 8: 707-25).

Alternatively, in another particular embodiment, the levels of the MEK protein, particularly MEK1 and/or MEK2 proteins are determined by Western blot. Western blot is based on the detection of proteins previously resolved by gel electrophoreses under denaturing conditions and immobilized on a membrane, generally nitrocellulose, by the incubation with an antibody specific and a developing system (e.g. chemiluminescent).

A MEK inhibitor may inhibit MEK activity, particularly MEK1 and/or MEK2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even 100% and all ranges between 5% and 100%. Suitable methods for determining whether an inhibitor acts by decreasing the MEK activity include any method which allows detecting a decrease in the phosphorylation of a key downstream effector of MEK, such as MAPK3/ERK1 and/or MAPK1/ERK2. Methods for detecting MEK activity are known in the art and include without limitation active MEK pulldown experiments, whole cell lysates immunoblotting, Enzyme Linked Immunosorbent Assays (ELISA) against the phosphorylated forms of both ERK1 and ERK2 (Garbison K E, Heinz B A, Lajiness M E, authors; Weidner J R, Sittampalam G S, editors. Phospho-ERK assays. 2012 May 1 [Updated 2015 Jun. 25]. In: Markossian S, Grossman A, Brimacombe K, et al., editors. Assay Guidance Manual [Internet]. Bethesda (MD): Eli Lilly & Company and the National Center for Advancing Translational Sciences; 2004), and luminescence-based immunoassays (Dixon A S. et al. 2016. ACS Chem Biol, 11(2):400-8). Commercial kits are available such as the MEKActivity Assay Kit (Sigma-Aldrich, Product Code: CS0490). Assays to determine the activity of an enzyme are known by the skilled person and include, without limitation, initial rate assays, progress curve assays, transient kinetics assays and relaxation assays. Continuous assays of enzymatic activity include, without limitation, spectrophotometric, fluorometric, calorimetric, chemiluminescent, light scattering and microscale thermophoresis assays. Discontinuous assays of enzymatic activity include, without limitation, radiometric and chromatographic assays. As the skilled person understands, factors that may influence enzymatic activity comprise salt concentration, temperature, pH, and substrate concentration.

MEK inhibitors can be any organic or inorganic molecule, including modified and unmodified nucleic acids such as antisense nucleic acids, RNA interference (RNAi) agents such as siRNA, shRNA or miRNA, peptides, proteins, peptidomimetics, receptors, ligands, antibodies and small organic molecules that are useful in treating cancer.

“Small chemical compound”, as used herein, relates to a molecule that regulates a biological process, in the present case inhibits catalytic activity of MEK. This compound can be natural or artificial.

Exemplary MEK inhibitors are disclosed in Mahapatra D K. et al. 2017. Expert Opinion on Therapeutic Patents, 27(8): 887-906.

In a preferred embodiment, MEK inhibitors useful in the present invention are selected from Table 1.

TABLE 1
MEK INHIBITORS SUITABLE FOR USE
ACCORDING TO THE INVENTION

| | |
|---|---|
| I | Trametinib/GSK1120212 (GlaxoSmithKline), Pimasertib (Santhera |
| | Pharmaceuticals), Selumetinib (Array BioPharma/AstraZeneca), |
| | Cobimetinib/XL518/GDC-0973 (Exelisis/Genentech), |
| | Binimetinib/MEK162 (Array BioPharma), Refametinib (Bayer), |
| | Mirdametinib/PD0325901, CI-1040/PD-184352 (Pfizer), RDEA-119 |
| | (Ardea Biosciences/Bayer), PD-0325901 (Pfizer), RO4987655 |
| | (Hoffman-La Roche), TAK733 (Millennium/Takeda), RO5126766 |
| | (Hoffman-La Roche), PD098059 (Parke-Davis/Pfizer), SL-327 |
| | (DuPont Pharmaceuticals), UO126 (DuPont Pharmaceuticals), |
| | RO5068760 (Hoffman-La Roche) |
| II | An interference RNA specific for the MEK sequence, particularly for |
| | the MEK1 and/or MEK2 sequence. |
| III | An antisense oligonucleotide specific for the MEK sequence, |
| | particularly for the MEK1 and/or MEK2 sequence. |
| IV | A ribozyme or DNA enzyme specific for the MEK sequence, |
| | particularly for the MEK1 and/or MEK2 sequence. |
| V | Inhibitory antibodies which bind specifically to MEK and inhibit MEK |
| | activity, particularly MEK1 and/or MEK2 activity. |
| VI | Aptamers and spiegelmers. |

In a preferred embodiment, the MEK inhibitor is selected from the group consisting of compounds listed in item I of Table I or a pharmaceutically acceptable salt thereof; more preferably is selected from the group consisting of Trametinib, Pimasertib, Selumetinib, Cobimetinib, Binimetinib, Refametinib, Mirdametinib, CI-1040/PD-184352, RDEA-119, PD-0325901, RO4987655, TAK733, RO5126766, PD098059, SL-327, U0126, RO5068760, and a pharmaceutically acceptable salt thereof.

In a more preferred embodiment, the MEK inhibitor is selected from the group consisting of Trametinib, Pimasertib, Selumetinib, Cobimetinib, Binimetinib, Refametinib, Mirdametinib, PD-0325901, CI-1040 and TAK-733; more preferably is selected from the group consisting of Trametinib, Pimasertib, Selumetinib, Cobimetinib, Binimetinib, Refametinib and Mirdametinib. In a more preferred embodiment, the MEK inhibitor is selected from the group consisting of trametinib, pimasertib and selumetinib. More preferably is trametinib.

All the compounds listed in item I of Table 1 also include pharmaceutically acceptable salts, solvates, polymorphs or cocrystals thereof. Prodrugs of these compounds are also included.

The term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

The term “pharmaceutically acceptable salt” embraces salts with a pharmaceutically acceptable acid or base. Pharmaceutically acceptable acids include both inorganic acids, for example and without limitation hydrochloric, sulfuric, phosphoric, diphosphoric, hydrobromic, hydroiodic and nitric acid and organic acids, for example and without limitation citric, fumaric, maleic, malic, mandelic, ascorbic, oxalic, succinic, tartaric, benzoic, acetic, methanesulphonic, ethanesulphonic, benzenesulphonic, cyclohexylsulfamic (cyclamic) or p-toluenesulphonic acid. Pharmaceutically acceptable bases include alkali metal (e.g. sodium or potassium) and alkali earth metal (e.g. calcium or magnesium) hydroxides and organic bases, for example and without limitation alkyl amines, arylalkyl amines and heterocyclic amines.

The term “solvate” according to this invention is to be understood as meaning any solid form of the above compounds which has another molecule attached to it via non-covalent bonding. Examples of solvates include hydrates and alcoholates, preferably C1-C6 alcoholates, e.g. methanolate.

The term “polymorph” according to this invention is to be understood as a specific crystalline form of a compound that can crystallize in different forms.

The term “cocrystal”, as used herein, is to be understood as a crystalline structure composed of a MEK inhibitor and at least another component. As used herein, the term “interference RNA” or “iRNA” refers to RNA molecules capable of silencing the expression of MEK, particularly MEK1 and/or MEK2 or of any gene needed for MEK function. To that end, iRNA are typically double-stranded oligonucleotides having at least 30 base pairs in length, and they more preferably comprise about 25, 24, 23, 22, 21, 20, 19, 18 or 17 ribonucleic acid base pairs. Several different types of molecules have been used effectively in iRNA technology including small interfering RNA (siRNA) sometimes known as short interference RNA or silencer RNA, micro RNA (miRNA) which normally differs from siRNA because they are processed from single-stranded RNA precursors and they are shown to be only partially complementary to the target mRNA and short hairpin RNA (shRNA).

Small interfering RNA (siRNA) agents are capable of inhibiting target gene expression by interfering RNA. siRNAs may be chemically synthesized, or may be obtained by in vitro transcription, or may be synthesized in vivo in target cell. Typically, siRNAs consist of a double-stranded RNA from 15 to 40 nucleotides in length and may contain a protuberant region 3′ and/or 5′ from 1 to 6 nucleotides in length. Length of protuberant region is independent from total length of siRNA molecule. siRNAs act by post-transcriptional degradation or silencing of target messenger.

siRNA may be denominated shRNA (short hairpin RNA) characterized because the antiparallel strands that form siRNA are connected by a loop or hairpin region. siRNAs are constituted by a short antisense sequence (19 to 25 nucleotides) followed by a loop of 5-9 nucleotides, and the sense strand. shRNAs may be encoded by plasmids or virus, particularly retrovirus and, more particularly, retrovirus and under the control of promoters such as U6 promoter for RNA polymerase Ill.

The siRNAs for use within the context of the invention are substantially homologous to MEK mRNA, particularly MEK1 and/or MEK2 mRNA, or its protein-coding genome sequence. The term “substantially homologous” is understood to mean that siRNAs have a sequence sufficiently complementary or similar to target mRNA so that siRNA may be able to provoke mRNA degradation by RNA interference. Suitable siRNAs to provoke interference include siRNAs formed by RNA, as well as siRNAs containing chemically different modifications such as:
siRNAs in which the links between nucleotides are different from those appearing in nature, such as phosphorothioate links;
stranded-RNA conjugates with a functional reagent, such as a fluorophoro;
modification of the ends of RNA strands, particularly the 3′ end by the combination with different functional hydroxyl groups at 2′-position;
sugar-modified nucleotides such as O-alkylated radicals at 2′-position such as 2′-O-methylribose or 2′-O-fluororibose;
base-modified nucleotides such as halogenated bases (for example 5-bromouracil and 5-iodouracil), alkylated bases (for example 7-methyl-guanosine).

The siRNAs and shRNAs for use within the context of the invention may be obtained using a series of techniques known to a person skilled in the art. For example, siRNA may be chemically synthesized from protected ribonucleoside phosphoramidites in a conventional DNA/RNA synthesizer. Alternatively, siRNA may be produced by recombinant dicer from plasmid and viral vectors, where the coding region of siRNA strand or strands is under operative control of RNA polymerase Ill promoters. RNase Dicer processes shRNA into siRNA in cells.

The MEK region which is taken as a basis for the design of siRNA is not limitative and may contain a region of coding sequence (between the initiation codon and the termination codon) or, alternatively, may contain sequences from the 5′ or 3′ untranslated region, preferably from 25 to 50 nucleotides in length and in any position in 3′ position with regard to the initiation codon. A procedure for siRNA design involves the identification of sequence motif AA(N19)TT wherein N may be any nucleotide in PARP sequence and the selection of those that exhibit a high content in G/C. If said sequence motif is not found, it is possible to identify sequence motif NA(N21) wherein N may be any nucleotide.

In a preferred embodiment the MEK inhibitor is an siRNA. In a more preferred embodiment the siRNA is a commercial siRNA from Santa Cruz Biotechnology, particularly sc-29396 or sc-35905.

In another embodiment, the inhibitor is an “antisense oligonucleotide” specific to MEK, i.e., molecules whose sequence is complementary to mRNA coding for MEK, particularly to MEK1 and/or MEK2, i.e., complementary to cDNA coding chain. The antisense oligonucleotide may be complementary to a complete coding region or a region of same including both the coding region and the 5′ and 3′ untranslated regions. The antisense oligonucleotides may consist of 5, 10, 15, 20, 25, 30, 35, 40, 45, 50 or more nucleotides in length. The antisense oligonucleotides may be obtained by chemical synthesis or by enzymatic binding reactions widely known to a person skilled in the art. For example, an antisense oligonucleotide may further contain modified nucleotides which increase its biological stability or the stability of the bicatenary DNA-RNA complexes formed between the antisense oligonucleotide and the target polynucleotide, such as phosphorothioate derivatives, peptide nucleic acids and acridine-substituted oligonucleotides. Modified oligonucleotides that may be used for the preparation of antisense nucleic acid include 5-fluorouracil, 5-bromouracil, 5-chlorouracil, 5-iodouracil, hypoxanthine, xanthine, 4-acetyl-citosine, 5-(carboxyhydroxylmethyl) uracil, 5-carboxymethylaminomethyl-2-thiouridine, 5-carboxymethyl-aminomethyl uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcitosine, 5-methylcitosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocitosine, 5-methyl-2-thiouracil, 2-thiouracil, 4-thiouracil, 5-methyluracil, uracil-5-oxyacetic acid methyl ester, 5-methyl-2-thiouracil, 3-(3-amino-3-N-2-carboxypropyl)uracil, and 2,6-diaminopurine. Alternatively, the antisense nucleic acid may be produced biologically using an expression vector in which the antisense-oriented nucleic acid has been cloned.

Another group of compounds that may form part of the present invention are catalytically active nucleic acids known as ribozimes. “Ribozimes” comprise a catalytic region and a second region whose sequence is complementary to target nucleic acid and confers substrate specificity on the ribozime. After the interaction between the ribozime and its substrate by hybridization and coupling between complementary regions of target nucleic acid and ribozime, an activation of the catalytic region is produced provoking the inter- or intramolecular rupture of target nucleic acid. Basic considerations for the design of ribozimes are widely known to a person skilled in the art (see, for example, Doherty and Doudna (Annu. Ref. Biophys. Biomolstruct. 2000; 30:457-75).

Another type of compounds that may form part of the compositions of the invention include inhibitory antibodies. The term “inhibitory antibody” is understood to mean, according to the present invention, an antibody that binds to MEK, particularly to MEK1 and/or MEK2, provoking the inhibition of its catalytic activity.

Antibodies may be prepared using any method known to a person skilled in the art. Thus, polyclonal antibodies are prepared by immunization of an animal with the protein aimed to be inhibited. Monoclonal antibodies can be prepared using the method described by Kohler, Milstein et al (Nature, 1975, 256: 495). Once antibodies capable of binding to MEK are identified, those antibodies capable of inhibiting MEK activity using the abovementioned assays for determination of MEK activity will be selected, particularly MEK1 and/or MEK2. Suitable antibodies in the present invention include intact antibodies which comprise an antigen-binding variable region and a constant region, fragments “Fab”, “F(ab′)2” y “Fab′”, Fv, scFv, diabodies and bispecific antibodies.

Other compounds capable of inhibiting MEK expression that may form part of the compositions of the invention include aptamers and spiegelmers. “Aptamers and spiegelmers” are single-stranded or double-stranded D- or L-nucleic acids that specifically bind to the protein resulting in a modification of the biological activity of protein (MEK, particularly MEK1 and/or MEK2). Aptamers and spiegelmers are 15 to 80 nucleotides in length and, preferably, 20 to 50 nucleotides in length.

In an embodiment, the combination of the invention is a conjugate between component (i) and component (ii) of the combination of the invention, particularly is a conjugate between a polypeptide comprising the sequence SEQ ID NO: 1 or a functionally equivalent variant thereof and a MEK inhibitor.

In some embodiments, a conjugation between components (i) and (ii) are via a noncleavable linker. In some embodiments, a conjugation between components (i) and (ii) are via a cleavable linker. Exemplary noncleavable linkers and cleavable linkers are described in U.S. Pat. Nos. 8,088,387, 8,142,784, WO2013075048, U.S. Pat. Nos. 6,630,579, 8,512,707, 9,120,854, 9,023,351, US20160095938, U.S. Pat. No. 9,446,146, WO2005009369, U.S. Pat. Nos. 5,773,001, 6,214,345, U.S. Ser. No. 10/111,954, U.S. Pat. Nos. 8,153,768, 7,829,531, US20160082119, WO2018218004, U.S. Pat. No. 8,568,728, WO2015057699, US20170182181, U.S. Pat. No. 9,198,979, the content of each of which is incorporated herein by reference in its entirety.

In another aspect, the invention relates to a pharmaceutical composition comprising a pharmaceutically effective amount of a combination of the invention together with a pharmaceutically acceptable excipient.

As it is used in the present invention, the expression “pharmaceutical composition” relates to a formulation that has been adapted for administering a predetermined dose of one or several therapeutic useful agents to a cell, a group of cells, an organ, a tissue or an animal in which cell division is uncontrolled, such cancer.

The pharmaceutical composition of the invention contains a pharmaceutically effective amount of a combination according to the invention and a pharmaceutically active carrier. The pharmaceutical composition of the invention comprises a polypeptide comprising the sequence SEQ ID NO: 1, a functionally equivalent variant thereof, a conjugate according to the invention, a polynucleotide encoding the polypeptide or the conjugate, a vector comprising the polynucleotide or a cell capable of secreting into the medium the polypeptide or the conjugate and a MEK inhibitor. Suitable functionally equivalent variants of the polypeptide of SEQ ID NO: 1, suitable conjugates, fusion proteins, polynucleotides, vectors or cells for use in the pharmaceutical composition according to the invention are as defined above.

The expression “pharmaceutically effective amount”, as used herein, is understood as an amount capable of providing a therapeutic effect, and which can be determined by the person skilled in the art by commonly used means. The amount of the Omomyc polypeptide, of the functionally equivalent variant thereof, of the conjugate, fusion protein, polynucleotide, vector, cell or of the MEK inhibitor that may be combined in the pharmaceutical compositions according to the invention will vary depending upon the subject and the particular mode of administration. Those skilled in the art will appreciate that dosages may also be determined with guidance from Goodman and Goldman's The Pharmacological Basis of Therapeutics, Ninth Edition (1996), Appendix II, pp. 1707-1711 and from Goodman and Goldman's The Pharmacological Basis of Therapeutics, Tenth Edition (2001), Appendix II, pp. 475-493.

The appropriate dosage of the active principle or principles within the pharmaceutical composition will depend on the type of cancer to be treated, the severity and course of the disease, whether the composition is administered for preventive or therapeutic purposes, previous therapy, the patient's clinical history and response to the peptide or polypeptide, and the discretion of the attending physician.

The amount of polypeptide comprising the sequence SEQ ID NO:1, the functionally equivalent variant thereof, the fusion protein, the conjugate, polynucleotide, vector or cell is suitably administered to the patient at one time or over a series of treatments. Depending on the type and severity of the disease, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day.

In a preferred embodiment, the amount of the first component is of about 3.75 mg/kg, of subject body weight per day, preferably administered four times per week, preferably intranasally administered. In a preferred embodiment, the amount of the first component is of about 8 to 15 mg/m², preferably of 10 to 12 mg/m², more preferably 11.25 mg/m² per day, preferably administered four times per week, preferably intranasally administered.

In a preferred embodiment, the amount of the first component is of about 50 mg/kg, of subject body weight per day, preferably administered twice per week, preferably intravenously administered. In a preferred embodiment, the amount of the first component is of about 100 to 200 mg/m², preferably of 125 to 175 mg/m², preferably of 140 to 160 mg/m², more preferably 150 mg/m² per day, preferably administered twice per week, preferably intravenously administered.

A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

In an embodiment, the combinations or compositions can be administered once a week, twice a week, three times a week, four times a week, five times a week, six times a week or seven times a week. In an embodiment, the combinations or compositions can be administered once a week. In another embodiment, the combinations or compositions can be administered twice per week. In another embodiment, the combinations or compositions can be administered four times a week. In another preferred embodiment, the first component of the combination or composition is administered four times a week and the second component of the combination or composition is administered once a week. In another embodiment, the first component of the combination or composition is administered twice per week and the second component of the combination or composition is administered once a week. Both compounds can be concomitantly administered or sequentially administered. When the compounds are sequentially administered, the administration of the first compound is discontinued before starting with the second compound.

The duration of the treatment can be at least one week, at least two weeks, at least three weeks, at least four weeks, at least five weeks, at least six weeks, at least seven weeks, at least eight weeks, at least nine weeks, at least ten weeks or more. Preferably, the duration of the treatment is at least four weeks. In another embodiment, the duration of the treatment is at least three weeks.

The amount of the MEK inhibitor depends on the specific agent used and may be about 0.01 mg/kg to about 200 mg/kg, preferably 0.01 mg/kg to about 150 mg/kg, more preferably 0.01 mg/kg to about 100 mg/kg, preferably 0.01 mg/kg to about 75 mg/kg, 0.01 mg/kg to about 50 mg/kg, more preferably 0.5 mg/kg to about 50 mg/kg, and preferably from about 1 mg/kg to about 25 mg/kg, of subject body weight per day, one or more times a day, to obtain the desired therapeutic effect, preferably administered orally. In another preferred embodiment, the amount of the MEK inhibitor is of about 0.01 mg/kg to about 5 mg/kg, preferably 0.01 mg/kg to about 3 mg/kg, preferably 0.01 mg/kg to about 2 mg/kg, more preferably 0.02 mg/kg to about 2 mg/kg, even more preferably 0.02 mg/kg to about 1.5 mg/kg of subject body weight per day, one or more times a day, preferably administered orally, to obtain the desired therapeutic effect. In another embodiment, the amount of the MEK inhibitor is of about 30 mg/m² to about 75 mg/m², preferably of about 50 mg/m² per day, more preferably of about 25 mg/m² per day, one or more times a day. In a preferred embodiment, the amount of the MEK inhibitor is of about 2.5 mg/kg, of subject body weight per day or 7.5 mg/m² per day, preferably administered once a week, more preferably administered daily, more preferably administered orally. In a preferred embodiment, the amount of the MEK inhibitor is of about 0.5 mg/kg, of subject body weight per day or 1.5 mg/m² per day, preferably administered once a week, more preferably administered daily, more preferably administered orally. In a preferred embodiment, the amount of the MEK inhibitor is of about 5 mg/kg, of subject body weight per day or 15 mg/m² per day, preferably administered once a week, preferably administered daily, more preferably administered orally. In another preferred embodiment, the amount of the MEK inhibitor is of about 10 mg/kg, of subject body weight per day, or 30 mg/m² per day, preferably administered once a week, preferably administered daily, more preferably administered orally. In another preferred embodiment, the amount of the MEK inhibitor is of about 50 mg/kg, of subject body weight per day, or 150 mg/m² per day, preferably administered once a week, preferably administered daily, more preferably administered orally. In another preferred embodiment, the amount of the MEK inhibitor is of about 100 mg/kg, of subject body weigh per day, or 300 mg/m² per day, preferably administered once a week, preferably administered daily, more preferably administered orally. The MEK inhibitor is preferably administered 7 days a week, preferably during 4 weeks.

The pharmaceutical compositions according to the invention, which contain a first component (i) selected from a polypeptide comprising SEQ ID NO:1, a functionally equivalent variant thereof, a fusion protein, a conjugate, a polynucleotide, a vector or a cell according to the invention and a second component (ii) which is a MEK inhibitor, may be presented as a single formulation (for example, as a tablet or a capsule comprising a fixed quantity of each one of the components) or can, on the other hand, be presented as separate formulations to be later combined for joint, sequential, or separate administration. The compositions of the invention also include the formulation as a kit-of-parts wherein the components are formulated separately but are packaged in the same container. Those skilled in the art will appreciate that the formulation of the different components in the pharmaceutical composition according to the invention may be similar, in other words, similarly formulated (in tablets or pills), which allows their administration by the same route. In the case where the different components of the invention are formulated separately, the two components can be presented in a blister. Each blister contains the drugs that must be consumed during the day. If the drugs must be administered several times a day, the drugs corresponding to each administration can be placed in different sections of the blister, preferably recording in each section of the blister the time of day when they should be administered. Alternatively, the components of the composition of the invention can be formulated differently so that the different components are differently administered. Thus, it is possible that the first component is formulated for its intravenous administration and the second component is formulated as a tablet or capsule for its oral administration or vice versa. The ratio between the components that are part of the combinations or pharmaceutical compositions according to the invention can be adjusted by the skilled person depending on the antitumor agent used in each particular case, as well as of the desired indication. Thus, the invention envisages compositions wherein the ratio between the quantities of component (i) and component (ii) can range from 50:1 to 1:50, in particular from 40:1 to 1:40, in particular from 30:1 to 1:30, in particular from 20:1 to 1:20, from 1:10 to 10:1, or from 5:1 to 1:5. In a more particular embodiment, the ratio between quantities ranges from 1:1 to 1:5, preferably from 1:1 to 1:3. In a more particular embodiment, the ratio ranges from 1:1 to 1:1.5, preferably from 1:1.3 to 1:1.4, more preferably 1:1.34. In another particular embodiment, the ratio ranges from 1:1 to 1:2.8, preferably from 1:2.6 to 1:2.7, more preferably 1:2.67. In another particular embodiment, the ratio between quantities ranges from 30:1 to 5:1, preferably from 30:1 to 8:1, more preferably from 25:1 to 15:1, more preferably from 20:1 to 10:1. In a particular embodiment the ratio between quantities ranges from 20:1 to 1:20. In an embodiment, the ratio is 20:1. In another embodiment the ratio is 1:20. In another embodiment, the ratio is 10:1. Exceptionally good results have been obtained when the ratio of component (i):component (ii), more preferably the ratio of Omomyc:trametinib, Omomyc:pimasertib or Omomyc:selumetinib; even more preferably the ratio of Omomyc:trametinib, ranges from 100:1 to 5,500,000:1; preferably from 1000:1 to 3,000,000:1; preferably from 4000:1 to 2,500,000:1; preferably from 4300:1 to 2,300,000:1; preferably from 4338.71:1 to 2,239,335.24:1; preferably from 5000:1 to 2,200,000:1; preferably from 10000:1 to 2,200,000:1; preferably from 20000:1 to 2,200,000:1; more preferably from 30000:1 to 2,200,000:1; more preferably from 34709.70:1 to 2,200,000:1; more preferably from 35000:1 to 2,200,000:1; more preferably from 50000:1 to 2,000,000:1; even more preferably from 100000:1 to 1,500,000:1; preferably from 500000:1 to 1,000,000:1; preferably from 750000:1 to 1,000,000:1. In a preferred embodiment the ratio of component (i):component (ii), more preferably the ratio of Omomyc:trametinib, Omomyc:pimasertib or Omomyc:selumetinib; even more preferably the ratio of Omomyc:trametinib, ranges from 4338.71:1 to 2,239,335.24:1; more preferably from 4500:1 to 2,200,000:1. In a preferred embodiment the ratio of component (i):component (ii), more preferably the ratio of Omomyc:trametinib, Omomyc:pimasertib or Omomyc:selumetinib; even more preferably the ratio of Omomyc:trametinib, ranges from 34,709.70:1 to 2,239,335.24:1; more preferably from 35000:1 to 2,200,000:1. Although these ratios are valid for treating any kind of cancer, more preferably, these ratios are obtained when a cancer selected from the group consisting of melanoma, colorectal cancer and breast cancer are treated, even more preferably when melanoma is treated, preferably a melanoma cancer mutated in BRAF, more preferably having a BRAF V600E mutation.

Preferably these ratios are w/w ratios.

The components of the pharmaceutical composition or the combination of the invention can be administered simultaneously. “Simultaneous administration” encompasses coadministration of the two therapeutic agents, regardless of the relative frequencies or timing of the administration of the respective agents. Thus, simultaneous administration encompasses the coadministration of the two therapeutic agents at the same time and at the same frequencies of administration. In addition, simultaneous administration refers to the coadministration of the two therapeutic agents, in which one agent is administered more frequently than the other(s). In addition, simultaneous administration refers to the coadministration of the two therapeutic agents, in which one agent is administered only once during the administration of the other agent(s).

In an embodiment component (i) is administered intranasally. In another embodiment component (i) is administered intravenously. In another embodiment, component (ii) is administered orally. In another embodiment, component (ii) is administered parenterally, particularly intraperitoneally or intravenously.

In a preferred embodiment component (i) of the combination or pharmaceutical composition of the invention is administered intravenously, whereas the MEK inhibitor is administered orally. For intravenous administration the preferred dose of component (i) of the combination or composition of the invention, preferably the preferred dose of the polypeptide or functionally equivalent variant thereof, fusion protein or conjugate ranges from 0.01 to 250 mg/Kg which can be administered in single or multiple doses, more preferably between 0.1 to about 100 mg/kg per day. The preferred dose of the MEK inhibitor for oral administration is 0.01 to 200 mg/Kg, preferably 0.01 to 150 mg/Kg, more preferable between 0.1 to 100 mg/Kg, more preferably 0.5 to 100 mg/Kg, even more preferably 10 to 100 mg/kg, most preferably 50 to 100 mg/Kg. In another preferred embodiment, the preferred dose of the MEK inhibitor for oral administration is 0.01 mg/kg to 5 mg/kg, preferably 0.01 mg/kg to 3 mg/kg, preferably 0.01 mg/kg to 2 mg/kg, more preferably 0.02 mg/kg to 2 mg/kg, even more preferably 0.02 mg/kg to 1.5 mg/kg. Preferably said MEK inhibitor is administered 7 days a week for 4 weeks.

In another embodiment components (i) and (ii) of the combination or pharmaceutical composition of the invention are administered intravenously.

The pharmaceutical composition of the invention can also contain one or several additional compounds for the prevention and/or treatment of pathologies in which there is an uncontrolled cell division, such as cancer. Said additional compounds, such as antitumoral agents can form part of the pharmaceutical composition as independent entities. In a preferred embodiment, the combinations or pharmaceutical compositions of the invention comprise one or more antitumoral agents selected from the group consisting of a cytotoxic agent, an antiangiogenic agent, an antimetastatic agent and an antiproliferative agent.

The pharmaceutical composition of the invention also contain one or several additional pharmaceutically acceptable excipients. “Pharmaceutically acceptable excipient” is understood a therapeutically inactive substance said to be used for incorporating the active ingredient and which is acceptable for the patient from a pharmacological/toxicological point of view and for the pharmaceutical chemist who manufactures it from a physical/chemical point of view with respect to the composition, formulation, stability, acceptation of the patient and bioavailability. The excipient can be a carrier. As used herein “carrier” is meant any substance that serves to improve the delivery and the effectiveness of the active principle within the pharmaceutical composition. In a preferred embodiment, the carrier does not allow direct delivery of component (i) and/or (ii) to the cytoplasm of the cells, i.e. the carrier is not capable of fusing with the plasmatic membrane of the target cells. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the combination or composition. Pharmaceutically acceptable carriers may further comprise minor amounts of auxiliary substances such as wetting or emulsifying agents, preservatives or buffers, which enhance the shelf life or effectiveness of components forming part of the combinations or compositions of the invention. Examples of proper carriers are well known in the literature (see for example Remington's Pharmaceutical Sciences, 19th ed., Mack Publishing Company, Easton, PA, 1995). Examples of carriers without limitation are a series of saccharide such as lactose, dextrose, sucrose, sorbitol, mannitol, xylitol, erythritol, and maltitol; a series of starch such as corn starch, wheat starch, rice starch, and potato starch; a series of cellulose such as cellulose, methyl cellulose, sodium carboxy methyl cellulose, and hydroxyl propylmethyl cellulose; and a series of filler such as gelatin and polyvinyl pyrrolidone. In some cases, a disintegrants such as cross-linked polyvinyl pyrrolidone, agar, alginic acid, or sodium alginate may be added.

The number and the nature of the pharmaceutically acceptable excipients depend on the desired dosage form. The pharmaceutically acceptable excipients are known by the person skilled in the art (Fauli y Trillo C. (1993) “Tratado de Farmacia Galénica”, Luzán 5, S.A. Ediciones, Madrid). Said compositions can be prepared by means of the conventional methods known in the state of the art (“Remington: The Science and Practice of Pharmacy”, 20th edition (2003) Genaro A. R., ed., Lippincott Williams & Wilkins, Philadelphia, US).

For pharmaceutical compositions comprising an agent that is a nucleic acid molecule, the nucleic acid molecule may be present within any of a variety of delivery systems known to those of ordinary skill in the art, including nucleic acid, and bacterial, viral and mammalian expression systems such as, for example, recombinant expression constructs as provided herein. Techniques for incorporating DNA into such expression systems are well known to those of ordinary skill in the art. The DNA may also be “naked,” as described, for example, in Ulmer et al., Science 259:1745-49, 1993 and reviewed by Cohen, Science 259:1691-1692, 1993. The uptake of naked DNA may be increased by coating the DNA onto biodegradable beads, which are efficiently transported into the cells.

Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland and Huang, Handb. Exp. Pharmacol. 137:165-92 (1999); Lee et al., ACS Symp. Ser. 752:184-92 (2000); U.S. Pat. No. 6,395,713; International Patent Application Publication No. WO 94/02595); Selbo et al., Int. J. Cancer 87:853-59 (2000); Selbo et al., Tumour Biol. 23:103-12 (2002); U.S. Patent Application Publication Nos. 2001/0007666, and 2003/077829). Such delivery methods known to persons having skill in the art, include, but are not restricted to, encapsulation in liposomes, by iontophoresis, or by incorporation into other vehicles, such as biodegradable polymers; hydrogels; cyclodextrins (see, e.g., Gonzalez et al., Bioconjug. Chem. 10: 1068-74 (1999); Wang et al., International Application Publication Nos. WO 03/47518 and WO 03/46185); poly(lactic-co-glycolic)acid (PLGA) and PLCA microspheres (also useful for delivery of peptides and polypeptides and other substances) (see, e.g., U.S. Pat. No. 6,447,796; U.S. Patent Application Publication No. 2002/130430); biodegradable nanocapsules; and bioadhesive microspheres, or by proteinaceous vectors (International Application Publication No. WO 00/53722). In another embodiment, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives (see also, e.g., U.S. Patent Application Publication No. 2003/0077829).

In a particular embodiment, when the composition or combination according to the invention comprises a nucleic acid (DNA, RNA, siRNA, antisense oligonucleotides, ribozimes, aptamers and spiegelmers), the pharmaceutical composition may be formulated as a composition intended for use in gene therapy; by way of illustration, not limitation, that pharmaceutical composition may contain a viral or nonviral vector, which comprises the suitable polynucleotide or gene construction. By way of illustration and not limitation, said vectors, may be viral, for example, based on retrovirus, adenovirus, etc., or nonviral such as ADN-liposome, ADN-polymer, ADN-polymer-liposome complexes, etc. [see “Nonviral Vectors for Gene Therapy”, edited by Huang, Hung and Wagner, Academic Press (1999)]. Said vectors, which contain the corresponding polynucleotide or gene construction, may be administered directly to a subject by conventional methods. Alternatively, said vectors may be used to transform, or transfect or infect cells, for example, mammal cells, including human, ex vivo, which subsequently will be implanted into a human body or an animal to obtain the desired therapeutic effect. For administration to a human body or an animal, said cells will be formulated in a suitable medium that will have no adverse influence on cell viability.

The combination or pharmaceutical composition of the invention can be administered by any type of suitable route, such as by oral route, topical route, by inhalation or parenteral route so that the pharmaceutically acceptable excipients necessary for the formulation of the desired dosage form will be included. Other routes of administration can be rectally, intracisternally or intravaginally. The preferred route of administration of said combination or pharmaceutical compositions is the intravenous route for component (i) and the oral route for component (ii). Alternatively, the route of administration of said combination or pharmaceutical compositions is the endovenous route for both components.

“Oral route” is understood as the pharmaceutical composition incorporated into the organism after deglutition. In a particular embodiment, the pharmaceutical composition of the invention can be in a dosage form suitable for its administration by oral route, whether it is solid or liquid. The dosage forms suitable for their administration by oral route can be tablets, capsules, syrups or solutions, and can contain any conventional excipient known in the art, such as binders, for example syrup, acacia, gelatin, sorbitol or polyvinylpyrrolidone; filling agents, for example lactose, sugar, corn starch, calcium phosphate, sorbitol or glycine; lubricants for compression, for example, magnesium stearate; disintegrating agents, for example starch, polyvinylpyrrolidone, sodium glycolate of starch or microcrystalline cellulose; or pharmaceutically acceptable wetting agents such as sodium lauryl sulfate. The solid oral compositions can be prepared by means of conventional processes of mixing, filling or compressing. Repetitive mixing operations can be used to completely distribute the active agent in those compositions that use high amounts of filling agents. Said operations are conventional in the art. The tablets can be prepared, for example, by means of wet or dry granulation, and optionally coating them according to the processes known in the common pharmaceutical practice, particularly with an enteric coating.

On the other hand, “topical route” is understood as an administration by non-systemic route, and includes the application of a pharmaceutical composition of the invention externally on the epidermis, in the oral cavity and the instillation of said composition into ears, eyes and nose, and in which it does not significantly enter the blood stream. Dosage forms for topical or transdermal administration of a compound of this invention include ointments, pastes, creams, lotions, gels, powders, solutions, sprays, inhalants or patches.

Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In an embodiment, the combination or pharmaceutical composition is administered systemically.

“Systemic route” is understood as the administration by oral route, intravenous route, intraperitoneal route and intramuscular route. The amount of components (i) and (ii) required for the therapeutic or prophylactic effect will naturally vary according to the elected compound, the nature and the severity of the illness that is going to be treated, and the patient. Preferably the combination or pharmaceutical composition is administered orally.

In another embodiment, the combination or pharmaceutical composition is administered intranasally. In a preferred embodiment, the intranasal administration is performed by instillation or nasal inhalation.

“Inhalation” is understood as the administration by intranasal route and by oral inhalation. The dosage forms suitable for said administration, such as a formulation in aerosol or a meter dosed inhaler can be prepared by means of conventional techniques. In an embodiment the route of administration is the intranasal route.

As it is used herein, the term “parenteral”, includes administration by intravenous route, intraperitoneal route, intramuscular route or subcutaneous route. Subcutaneous, intramuscular and intravenous dosage forms of parenteral administration are generally preferred. In an embodiment, the combination or pharmaceutical composition is administered intravenously.

In one embodiment, the combinations or pharmaceutical compositions of the invention can be adapted for their parenteral administration, such as sterile solutions, suspensions or lyophilized products in the appropriate dosage unit form. The combinations or pharmaceutical compositions suitable for its injectable use include sterile aqueous solutions (when they are soluble in water), or dispersions and sterile powders for the extemporaneous preparation of sterile injectable solutions or dispersions. For its administration by intravenous route, some suitable carriers include saline solution buffered with phosphate (PBS). In all the cases, the combination or composition must be sterile, and must be fluid to the point which that there exists easy ability for being injected. It must be stable in the preparation and storage conditions, and must be protected from the contamination action of microorganisms such as bacteria and fungi. The carrier can be a solvent or a dispersion medium which contains, for example, water, ethanol, a pharmaceutically acceptable polyol such as glycerol, propylene glycol, liquid polyethylene glycol and suitable mixtures thereof. Suitable fluidity can be maintained, for example, by means of using a coating such as lecithin, by means of maintaining the particle size required in the case of dispersion and by means of using surfactants. The prevention of the action of the microorganisms can be achieved by means of various antibacterial and antifungal agents, for example, parabens, chlorobutanol, phenol, ascorbic acid, thiomersal, and the like. In most cases, it will be preferable to include isotonic agents, for example, sugars; polyalcohols such as mannitol, sorbitol; or sodium chloride in the composition. The prolonged absorption of the injectable compositions may be caused by the inclusion of an agent which delays the absorption, for example, aluminum and gelatin monostearate.

The injectable sterile solutions can be prepared by incorporating the active compound in the required amount in a suitable solvent with one or a combination of the aforementioned ingredients, as needed, followed by sterilization by filtration through sterile membranes. Generally, the dispersions are prepared by incorporating the active compound in a sterile vehicle containing a basic dispersion medium and the rest of the ingredients required from among those previously listed. In the case of sterile powders for the preparation of injectable sterile solutions, the preferred preparation processes are vacuum drying and lyophilization which give rise to a powder with the active ingredient plus any desired additional ingredient from a previously filtered sterile solution thereof.

The combinations or pharmaceutical compositions of the invention can be suitably administered by means of pulse infusion, for example, with decreasing doses of the composition. Preferably, the dose is administered by means of injections, more preferably intravenous or subcutaneous injections, partly depending if the administration is acute or chronic.

Alternatively, as mentioned above, the different components of the composition are differently administered.

Thus, in an embodiment component (i) of the combination or composition, preferably the polypeptide or functionally equivalent variant or the conjugate of the invention, is administered intravenously while the MEK inhibitor is administered orally.

In another embodiment both components (i) and (ii) are administered intravenously.

In another embodiment, component (i) of the combination or composition, preferably the polypeptide or functionally equivalent variant thereof, or the conjugate of the composition, is administered intranasally or by inhalation.

Dosage forms of compositions intended for intranasal and intrapulmonary administration are preferably a liquid, a suspension or a solid. A suspension is a liquid preparation containing solid particles dispersed in a liquid vehicle. The dosage forms are preferably metered. For example, metered drops/sprays mean that the dispenser that includes the drops/spray delivers the drops/spray containing a metered dose (a predetermined quantity) of the composition for use according to the invention.

One preferred dosage form in the context of the intranasal administration route includes nasal drops. Drops are deposited mostly in the posterior portion of the nose and thus removed rapidly into the nasal pharynx. A concern with drops is often how to precisely control the drug's dose which is particularly important for the administration of the composition.

Another intranasal dosage form by which the pharmaceutical composition of the invention can be administered is nasal sprays. Nasal sprays typically contain the conjugate dissolved or suspended in a solution or a mixture of excipients (e.g. preservatives, viscosity modifiers, emulsifiers, buffering agents) in a non-pressurized dispenser. Nasal sprays have several advantages including compactness of the delivery device, convenience, simplicity of use, and accuracy of delivering dosages of 25 to 200 pL. They are deposited in the anterior portion of the nose and cleared slowly into nasal pharynx by mucociliary clearance. The nasal spray as used herein can be a liquid or a suspension.

Another intranasal dosage form is a nasal aerosol. Nasal aerosols differ from nasal sprays by the method of the composition dispensing: in aerosols, a compound is dispensed due to an excess of pressure and releases through a valve. In sprays, a compound is dispensed due to forcing away by a micropump bucket, while the pressure in the vial is similar to atmosphere pressure. Aerosols have similar advantages as sprays.

The composition according to the invention may alternatively preferably be administered by nasal emulsions, ointments, gels, pastes or creams. These are highly viscous solutions or suspensions applied to the nasal mucosa.

Due to the limited volume of the composition that can be efficiently delivered to the nasal mucosa, liquid intranasal dosage forms usually have higher concentrations as the corresponding intravenous dosage forms. When substances become poorly soluble or are instable in liquid form, powders can be used to administer the composition of the invention. Further advantages of powders are that they do not require preservatives and have usually a higher stability as compared to liquid formulations. The main limitation on intranasal powder application is related to its irritating effect on the nasal mucosa.

One dosage form in context of intrapulmonary administration is an inhalation aerosol. Inhalation aerosols are usually packaged under pressure and contain the composition according to the invention which is released upon activation of a valve system into the respiratory tract, in particular the lungs. The released aerosol is a colloid of fine solid particles (suspension) or liquid droplets (solution) in air or another gas. Accordingly, the aerosol may be a solution or a suspension aerosol. The liquid droplets or solid particles have preferably a diameter of less than 100 pm, more preferably less than 10 pm, most preferably less than 1 pm.

Another dosage form in context of intrapulmonary administration is inhalation sprays. Inhalation sprays are typically aqueous based and do not contain any propellant. They deliver the conjugate to the lungs by oral inhalation.

Nebulized inhalation solutions and suspensions may also be used to deliver the conjugate by the intrapulmonary route. Nebulized inhalation solutions and suspensions are typically aqueous-based formulations that contain the composition according to the invention. The nebulized inhalation solutions and suspensions deliver the composition to the lungs by oral inhalation for systemic effects and are used with a nebulizer.

Dry powder inhalation is an alternative to aerosol inhalation. The composition is usually included in a capsule for manual loading or within the inhaler. Dry powders are typically delivered by an inhaler to the lungs by oral inhalation. The dry powders as used herein can be formulated neat. Neat formulations contain the drug alone or quasi-alone e.g. as spry dried powder. The dry powders as used herein can be also formulated with a carrier such as lactose.

Intrapulmonary dosage forms are preferably metered, i.e. are delivered to the lungs in a predetermined quantity.

Devices for intranasal delivery in the context of the present invention include spray pump systems, pipettes for delivering drops, metered-dose spray pumps, nasal pressurized metered-dose inhalers, powder spray systems, breath-actuated powder inhalers and nasal powder insufflators. The intranasal delivery device may be filled with a single dose amount or a multi-dose amount of the intranasal formulation.

Using the intrapulmonary route the conjugate may be administered with a metered dose inhaler. A metered-dose inhaler (MDI) provides a fine mist of conjugate, generally with an aerodynamic particle size of less than 5 pm.

Dry powder inhalers can be alternatively used to deliver the composition intrapulmonary. Dry powder inhalers present powders as single-dose or multidose powders.

Another device for intrapulmonary delivery is a nebulizer including ultrasonic and air jet nebulizers. In ultrasonic nebulizers, ultrasound waves are formed in an ultrasonic nebulizer chamber by a ceramic piezoelectric crystal that vibrates when electrically excited. This generates an aerosol cloud at the solution surface. The aerosol produced by an air jet nebulizer is generated when compressed air is forced through an orifice. A liquid may be withdrawn from a perpendicular nozzle (the Bernoulli Effect) to mix with the air jet which is atomized using baffles to facilitate the formation of the aerosol cloud.

In one embodiment, each of the components of the combination or the pharmaceutical composition of the invention is prepared with carriers which will protect the components, particularly component (i), from a rapid elimination from the body, such as a controlled release formulation, including implants and microencapsulated administration systems.

Biodegradable biocompatible polymers such as ethylene vinylacetate, polyanhydrides, polyglycolic acid, collagen, polyorthoesters and polylactic acid can be used. The processes for preparing said formulations will be clear for persons skilled in the art. The materials can also be commercially obtained in Alza Corporation and Nova Pharmaceuticals, Inc.

The sustained release compositions also include preparations of crystals suspended in suitable formulations which can maintain the crystals in suspension. These preparations, when they are injected by subcutaneous or intraperitoneal route may produce a sustained release effect. Other compositions also include the components (i) and/or (ii) trapped in liposomes. The liposomes containing such components are prepared by means of known methods such as Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949. In a preferred embodiment, components (i) and/or (ii) are contained in liposomes, preferably both components are contained in liposomes, more preferably in the same liposome.

Despite the fact that Omomyc, functionally equivalent variants thereof, conjugates and fusion proteins of the invention are capable of translocating across biological membranes, it is possible to formulate Omomyc, any of its functionally equivalent variants, conjugates, polynucleotides, vectors or cells in nanoparticles. The nanoparticles may contribute to preserve the integrity of the components in the biological fluids until it reaches the target organ. Moreover, in the case of compositions comprising component (ii) or other antitumor agent, encapsulation of the composition may decrease secondary effects caused by the antitumor agent. In addition, nanoparticles can also be modified so as to include moieties which allow the targeting of the nanoparticle to an organ of interest. In this way, component (i) of the combination or the composition of the invention will be delivered in the proximity of the target organ, facilitating access of component (i) to the interior of the cells where its biological activity is required.

Thus, in another embodiment, component (i) of the combination or composition of the invention is provided forming part of a nanoparticle. In another embodiment, both components of the combination or composition of the invention are provided forming part of a nanoparticle, preferably both components are provided inside the same nanoparticle.

As used herein, the term “nanoparticle” refers to any material having dimensions in the 1-1,000 nm range. In some embodiments, nanoparticles have dimensions in the 2-200 nm range, preferably in the 2-150 nm range, and even more preferably in the 2-100 nm range. Nanoparticles that can be used in the present invention include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube. Molecules can be either embedded in the nanoparticle matrix or may be adsorbed onto its surface, preferably molecules are embedded in the nanoparticle.

In a preferred embodiment, the nanoparticle is a liposome.

Targeted delivery can be achieved by the addition of ligands without compromising the ability of nanoparticles to deliver their content. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with a nanoparticle, and can be conjugated to the nanoparticles by a variety of methods as discussed herein.

Examples of proteins or peptides that can be used to target nanoparticles include transferin, lactoferrin, TGF-β, nerve growth factor, albumin, HIV Tat peptide, RGD peptide, and insulin, as well as others.

It will be understood that the formulations of the invention in a nanoparticle are not intended or are not solely intended for facilitating the access of the components (i) and/or (ii) to the interior of the cell but to protect components (i) and/or (ii) from degradation and/or for facilitating targeting of the nanoparticle to the organ of interest.

In one example, the nanoparticle may be made up of a biodegradable polymer such as poly(butylcyanoacrylate) (PBCA). Examples of elemental nanoparticles include carbon nanoparticles and iron oxide nanoparticles, which can then be coated with oleic acid (OA)-Pluronic®. In this approach, a drug (e.g., a hydrophobic or water insoluble drug) is loaded into the nanoparticle. Other nanoparticles are made of silica.

Nanoparticles can be formed from any useful polymer. Examples of polymers include biodegradable polymers, such as poly(butyl cyanoacrylate), poly(lactide), poly(glycolide), poly-s-caprolactone, poly(butylene succinate), poly(ethylene succinate), and poly(p-dioxanone); poly(ethyleneglycol); poly-2-hydroxyethylmethacrylate (poly(HEMA)); copolymers, such as poly(lactide-co-glycolide), poly(lactide)-poly(ethyleneglycol), poly(poly(ethyleneglycol)cyanoacrylate-cohexadecylcyanoacrylate, and poly [HEMA-co-methacrylic acid]; proteins, such as fibrinogen, collagen, gelatin, and elastin; and polysaccharides, such as amylopectin, a amylose, and chitosan.

Other nanoparticles include solid lipid nanoparticles (SLN). Examples of lipid molecules for solid lipid nanoparticles include stearic acid and modified stearic acid, such as stearic acid-PEG 2000; soybean lecithin; and emulsifying wax. Solid lipid nanoparticles can optionally include other components, including surfactants, such as Epicuron® 200, poloxamer 188 (Pluronic® F68), Brij 72, Brij 78, polysorbate 80 (Tween 80); and salts, such as taurocholate sodium. Agents can be introduced into solid lipid nanoparticles by a number of methods discussed for liposomes, where such methods can further include high-pressure homogenization, and dispersion of microemulsions.

Nanoparticles can also include nanometer-sized micelles. Micelles can be formed from any polymers described herein. Exemplary polymers for forming micelles include block copolymers, such as poly(ethylene glycol) and poly(ε-caprolactone). (e.g., a PEO-b-PCL block copolymer including a polymer of ε-caprolactone and α-methoxy-ω-hydroxy-poly(ethylene glycol)).

In certain embodiments, the properties of the nanoparticles are altered by coating with a surfactant. Any biocompatible surfactant may be used, for example, polysorbate surfactants, such as polysorbate 20, 40, 60, and 80 (Tween 80); Epicuron® 200; poloxamer surfactants, such as 188 (Pluronic® F68) poloxamer 908 and 1508; and Brij surfactants, such as Brij 72 and Brij 78.

Nanoparticles can optionally be modified to include hydrophilic polymer groups (e.g., poly(ethyleneglycol) or poly(propyleneglycol)), for example, by covalently attaching hydrophilic polymer groups to the surface or by using polymers that contain such hydrophilic polymer groups (e.g., poly[methoxy poly (ethyleneglycol) cyanoacrylate-co-hexadecyl cyanoacrylate]). Nanoparticles can be optionally cross linked, which can be particularly useful for protein-based nanoparticles.

In another embodiment, the pharmaceutical composition of the invention is a nanoemulsion. “Nanoemulsion” as used herein means a colloidal dispersion of droplets (or particles) which at least some of the droplets have diameters in the nanometer size range. The nanoemulsions are comprised of omega-3, -6 or -9 fatty acid rich oils in an aqueous phase and thermo-dynamically stabilized by amphiphilic surfactants, which make up the interfacial surface membrane, produced using a high shear microfluidization process usually with droplet diameter within the range of about 80-220 nm.

### Therapeutic Uses of the Invention

In an aspect, the invention relates to the combination or the pharmaceutical composition of the invention for use in medicine.

In a further aspect, the invention relates to the combination or the pharmaceutical composition of the invention for use in the prevention and/or treatment of cancer.

In another aspect, the invention refers to the combination or the pharmaceutical composition of the invention for the preparation of a medicament for the prevention and/or treatment of cancer.

In another aspect, the invention also refers to a method for the prevention and/or treatment of cancer that comprises administering to a subject in need thereof a therapeutically effective amount of the combination or pharmaceutical composition of the invention.

In a preferred embodiment, the preventive or therapeutic method according to the invention involves the direct use of a combination or composition comprising a polypeptide comprising Omomyc, a functionally equivalent variant thereof, a conjugate or a fusion protein. Thus, in a preferred embodiment, the preventive or therapeutic methods according to the invention do not involve the administration of the nucleic acid encoding a polypeptide comprising Omomyc or the functionally equivalent variant thereof or fusion protein, or the administration of a vector encoding this nucleic acid, or a cell comprising said nucleic acid.

“Prevention” is understood as the administration of a combination or composition of the invention in an initial or early stage of the disease, or to also prevent its onset.

The term “treatment” is used to designate the administration of a combination or composition of the invention to control the progression of the disease before or after the clinical signs have appeared. Control of the progression of the disease is understood as the beneficial or desired clinical results which include but are not limited to reduction of the symptoms, reduction of the duration of the disease, stabilization of pathological conditions (specifically avoiding additional impairment), delaying the progression of the disease, improving the pathological condition and remission (both partial and complete). The control of the progression of the disease also involves a prolongation of survival in comparison to the expected survival if the treatment was not applied. In a preferred embodiment, control of the progression of the disease is measured as healthy lung/thorax volume ratio. In another embodiment, control of the progression of the disease is measured as reduction in tumor volume. In another embodiment, control of the progression of the disease is measured as reduction in tumor cell viability.

The term “cancer” is referred to a disease characterized by uncontrolled cell division (or by an increase of survival or apoptosis resistance), by the ability of said cells to invade other neighbouring tissues (invasion) or by the spread to other areas of the body where the cells are not normally located (metastasis) through the lymphatic and blood vessels. Depending on whether or not tumours can spread by invasion and metastasis, they are classified as being either benign or malignant: benign tumours are tumours that cannot spread by invasion or metastasis, i.e., they only grow locally; whereas malignant tumours are tumours that are capable of spreading by invasion and metastasis. The methods according to the present invention are useful for the treatment of local and malignant tumours.

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), hairy cell leukemia, polycythemia vera, lymphoma (e.g., Hodgkin's disease or non-Hodgkin's disease), AIDS-associated leukemias, Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, mendotheliosarcoma, lymphangiosarcoma, lymphangioendotheliosarcoma, synovioma, mesothelioma, Ewing's tumor, leiomyosarcoma, rhabdomyosarcoma, Kaposi's sarcoma, colon carcinoma, pancreatic cancer, breast cancer, biliary tract cancer, esophageal cancer, ovarian cancer, prostate cancer, oral cancer including squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, teratoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, intraepithelial neoplasms including Bowen's disease and Paget's disease, neuroglioma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-1 in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892).

Other cancers will be known to one of ordinary skill in the art.

In a preferred embodiment, the cancer is selected from the group consisting of breast cancer, ovarian cancer, pancreatic cancer, prostate cancer, lung cancer, colorectal cancer, stomach/gastric cancer, endometrial/uterine/cervical cancer, bladder cancer, head and neck cancer, leukemia, sarcoma, cholangiocarcinoma, glioblastoma, multiple myeloma, lymphoma. More preferable, the cancer is selected from the list consisting of breast cancer, ovarian cancer, and prostate cancer, even preferably it is a breast cancer or an ovarian cancer, yet more preferably it is a breast cancer.

In a more preferred embodiment, the cancer is selected from the group consisting of melanoma, colorectal cancer and breast cancer.

In a preferred embodiment, the cancer is a BRAF mutant cancer, i.e., a cancer having a BRAF mutation. More preferably, is a BRAF V600E mutant cancer.

In another embodiment, the mutation in BRAF is selected from the group consisting of a BRAF-wild type amplification, a BRAF deletion, a mutation in the BRAF gene that is a mutation at the valine at position 600, at the arginine at position 462, at the isoleucine at position 463, at the glycine at position 464, at the glycine at position 466, at the serine at position 467, at the glycine at position 469, at the aspartic at position 594, at the glycine at position 596, at the leucine at position 597, at the threonine at position 599, or at the lysine at position 601. Preferably, the mutation is at the valine at position 600. In a more preferred embodiment, the mutation is selected from the group consisting of the V600E mutation, the V600K mutation, the V600D mutation, the V600R mutation, the R4621 mutation, the 1463S mutation, the G464E mutation, the G466E mutation, the G466V mutation, the S467L mutation, the G469A mutation, the G469R mutation, the G469V mutation, the D594G mutation, the D594N mutation, the G596R mutation, the L597R mutation, the L597V mutation, the K601E mutation, the K601 D mutation, and the K601R mutation. In a preferred embodiment, the BRAF mutation is the V600E mutation.

In another embodiment, the cancer to be treated is a wild-type BRAF cancer.

In a preferred embodiment, the cancer is melanoma cancer. The term “melanoma” relates to malignant melanoma, and is a type of skin cancer that develops from melanocytes. More preferably, the melanoma has a BRAF mutation.

In a preferred embodiment, the cancer is colorectal cancer. Preferably, is a colorectal cancer having a BRAF mutation.

In a preferred embodiment, the cancer is breast cancer. The term “breast cancer” relates to any malignant proliferative disorder of breast cells, most commonly from the inner lining of milk ducts or the lobules that supply the ducts with milk. Cancers originating from ducts are known as ductal carcinomas, while those originating from lobules are known as lobular carcinomas. Preferably, the breast cancer has a BRAF mutation.

In a preferred embodiment the cancer is triple negative breast cancer (TNBC). The term triple negative breast cancer refers to the fact that the cancer cells are immunohistochemically defined by the lack of expression of estrogen receptor (ER), progesterone receptor (PR) and human epidermal growth factor receptor 2 (HER2), i.e., the cells are negative on all 3 tests. It is a highly malignant subtype of breast cancer usually associated with relatively poor clinical outcomes, earlier recurrence, and high propensity for visceral metastases than other breast cancer types.

In another embodiment, the cancer is pancreatic cancer, particularly is pancreatic ductal adenocarcinoma.

In another embodiment, the cancer is glioblastoma.

“Glioblastoma”, also known as glioblastoma and grade IV astrocytoma, is the most common and most aggressive cancer that begins within the brain.

In another embodiment, the cancer is lung cancer.

The terms “lung cancer” or “lung tumour” refer to the physiological condition in mammals characterized by unregulated cell growth in tissues of the lung. The term lung cancer is meant to refer to any cancer of the lung and includes non-small cell lung carcinomas and small cell lung carcinomas. In an embodiment, the lung cancer is non-small cell lung cancer (NSCLC). In another embodiment, the lung cancer is small cell lung cancer (SCLC).

The term non-small cell lung cancer (NSCLC), as used herein, refers to a group of heterogeneous diseases grouped together because their prognosis and management is roughly identical and includes, according to the histologic classification of the World Health Organization/International Association for the Study of Lung Cancer (Travis W D et al. Histological typing of lung and pleural tumours. 3rd ed. Berlin: Springer-Verlag, 1999):
(i) squamous cell carcinoma (SCC), accounting for 30% to 40% of NSCLC, starts in the larger breathing tubes but grows slower meaning that the size of these tumours varies on diagnosis.
(ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment.
(iii) large cell carcinoma is a fast-growing form that grows near the surface of the lung. It is primarily a diagnosis of exclusion, and when more investigation is done, it is usually reclassified to squamous cell carcinoma or adenocarcinoma.
(iv) adenosquamous carcinoma is a type of cancer that contains two types of cells: squamous cells (thin, flat cells that line certain organs) and gland-like cells.
(v) carcinomas with pleomorphic, sarcomatoid or sarcomatous elements. This is a group of rare tumours reflecting a continuum in histologic heterogeneity as well as epithelial and mesenchymal differentiation.
(vi) carcinoid tumour is a slow-growing neuroendocrine lung tumour and begins in cells that are capable of releasing a hormone in response to a stimulus provided by the nervous system.
(vii) carcinomas of salivary gland type begin in salivary gland cells located inside the large airways of the lung.
(viii) unclassified carcinomas include cancers that do not fit into any of the aforementioned lung cancer categories.

In a particular embodiment, the NSCLC is selected from squamous cell carcinoma of the lung, large cell carcinoma of the lung and adenocarcinoma of the lung.

The term small cell lung cancer (SCLC), as used herein, refers to a proliferation of small cells with unique and strict morphological features, containing dense neurosecretory granules which give this tumor an endocrine/paraneoplastic syndrome associated. Most cases arise in the larger airways (primary and secondary bronchi). These cancers grow quickly and spread early in the course of the disease.

In an even more preferred embodiment, the lung cancer is adenocarcinoma, more preferably a BRAF-driven lung adenocarcinoma, preferably a cancer associated with a mutation in the BRAF gene. Any of the mutations disclosed above can be present in the lung cancer.

In another preferred embodiment, the cancer to be treated according to the invention is characterized by expressing increased levels of MEK, particularly MEK1 and/or MEK2. MEK levels, particularly MEK1 and/or MEK2 levels are considered to be increased with respect to a reference value when the levels of MEK, particularly MEK1 and/or MEK2 in a sample of said cancer show an increase of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150% or more. The reference value can be the value corresponding to the level of expression of MEK, particularly MEK1 and/or MEK2 in a non-cancer sample.

In an embodiment, the cancer is a primary tumor. The term “primary tumor”, as used herein, refers to a tumor that originated in the location or organ in which it is present and did not metastasize to that location from another location.

In another embodiment, the cancer is a cancer metastasis. In the context of the present invention, “metastasis” is understood as the propagation of a cancer from the organ where it started to a different organ. It generally occurs through the blood or lymphatic system. When the cancer cells spread and form a new tumor, the latter is called a secondary or metastatic tumor. The cancer cells forming the secondary tumor are like those of the original tumor. If a breast cancer, for example, spreads (metastasizes) to the lung, the secondary tumor is formed of malignant breast cancer cells. The disease in the lung is metastatic breast cancer and not lung cancer. The authors of the present invention have also observed that the combination or composition of the invention is capable of decreasing cell proliferation irrespective of whether the cancer shows increased expression or activity of the Myc protein. In a preferred embodiment, the cancer to be prevented or treated is Myc-induced cancer.

In an embodiment, the cancer is a solid tumour.

In another embodiment, the cancer is a cancer resistant to MEK inhibitors.

“Resistance” is the reduction in effectiveness of a medication in treating a disease or condition. The expression “cancer resistant”, a used herein, refers to a cancer having resistance to MEK inhibitors either innate or acquired resistance. Innate resistance occurs when MEK inhibitors are ineffective from the start of treatment, due to pre-existing resistance mechanisms. Acquired resistance occurs when MEK inhibitors become ineffective during the course of treatment and after a clinical benefit has been observed.

All combinations of compounds of the invention and types of cancer are included in the present invention.

In some embodiments, the combination or composition of the invention produces an arresting of the growth of the tumor. In some embodiments, the combination or composition of the invention produces a reduction in the tumor size (e.g., volume or mass) by at least 5%, 10%, 25%, 50%, 75%, 90% or 99% relative to the size of the tumor prior to treatment.

In some embodiments, the combination or composition of the invention produces the reduction of the quantity of the tumor in the patient by at least 5%, 10%, 25%, 50%, 75%, 90% or 99% relative to the quantity of the tumor prior to treatment.

A “subject”, as used herein, includes any animal that has a cancer or exhibits a symptom of cancer, or is at risk for having a cancer or exhibiting a symptom of cancer. Suitable subjects (patients) include laboratory animals (such as mouse, rat, rabbit, or guinea pig), farm animals, and domestic animals or pets (such as cats or dogs). Non-human primates and, preferably, human patients, are included. Preferably, the subject is a mammal, most preferably a human.

The combinations or compositions for use in the prevention and/or treatment of cancer, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In a preferred embodiment, component (i) of the invention, preferably the polypeptide or the functionally equivalent variant thereof, or the conjugate, synergistically interact with the MEK inhibitor of the combination or composition in treating cancer (to achieve the therapeutic effect).

Particularly, in a more preferred embodiment, the combination or pharmaceutical composition for use in the prevention and/or treatment of cancer is a combination or pharmaceutical composition wherein the polypeptide or the functionally equivalent variant thereof, or the conjugate, is in an amount that synergistically interact with the MEK inhibitor in treating cancer.

The terms “synergistic effect” or “synergistically interact” are used interchangeably. A synergistic effect is one that is greater than the additive effect that would be predicted by summing the actual effects of the individual agents in vitro. In vivo, a synergistic effect is a physiological effect, and particularly a therapeutic effect, that is greater than the additive effect that would be predicted by summing the actual effects of the individual agents in vivo.

Thus, if two agents are administered, they together provide a measurable physiological effect, and particularly a therapeutic effect, if the actual effect of the agents together is greater than would be predicted by summing the actual therapeutic effects of the individual agents. Particularly, a synergistic effect is provided when a first agent alone provides some measurable effect, a second agent alone provides some measurable effect, and together the two agents provide a measurable effect greater than the effect provided by the sum of both individual agents. More particularly, a synergistic effect is provided when a first agent alone provides no measurable effect, a second agent alone provides some measurable effect, and together the two agents provide a measurable effect greater than the effect provided by the second agent alone. Still more particularly, a synergistic effect is provided when neither a first agent alone nor a second agent alone provide any measurable effect, but together the two agents provide a measurable effect. Since components (i) and (ii) act synergistically, the amount of components (i) and/or (ii) of the combinations or compositions of the invention may be less than that required in a monotherapy utilizing only one of them as a therapeutic agent. Preferably, in these combinations or compositions, a dosage of between 0.01-1.000 μg/kg body weight/day of the one or the other therapeutic agent can be administered.

In a preferred embodiment, the degree of synergy is calculated by the Zero interaction potency (ZIP) model (Bhagwan Y. et al. 2015. Comput Struct Biotechnol J, 13: 504-513). In a more preferred embodiment, the ZIP synergy scores were calculated using SynergyFinder 3.0 web application derived from Ianevsky A et al. Nucleic Acids Res. 2022; 50(W1):W739-W743.

The amount of the therapeutic agents present in the combinations or compositions may be no more than the amount that would normally be administered in a composition comprising that therapeutic agent as the only active agent. Preferably, the amount of a therapeutic agent in the present compositions will range from about 50% to 100% of the amount normally present in a composition comprising that agent as the only therapeutically active agent. In some embodiments, one therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The combination or composition of the invention may also be used in combination with known therapeutic processes, for example, in combination with chemotherapy, radiotherapy, immunotherapy, phototherapy, surgical intervention, hormones, or a combination of these.

All the embodiments of the combination of the invention are also applicable to the therapeutic methods of the invention.

### Articles of Manufacture and Kits

The disclosure also provides articles of manufacture comprising any one of the combinations or the pharmaceutical compositions disclosed herein, in one or more containers. In some embodiments, the article of manufacture comprises, e.g., a brochure, printed instructions, a label, or package insert directing the user (e.g., a distributor or the final user) to combine and/or use the compositions of the article of manufacture for the prevention and/or treatment of cancer.

In some embodiments, the article of manufacture comprises, e.g., bottle(s), vial(s), cartridge(s), box(es), syringe(s), injector(s), or any combination thereof. In some embodiments, the label refers to use or administration of the combinations or the pharmaceutical compositions in the article of manufacture according to the methods disclosed herein. In some aspects, the label suggests, e.g., a regimen for use, a regimen for treating, preventing, or ameliorating a cancer.

The contents of all cited references (including literature references, patents, patent applications, and websites) that may be cited throughout this application are hereby expressly incorporated by reference in their entirety for any purpose, as are the references cited therein.

All terms as used herein, unless otherwise stated, shall be understood in their ordinary meaning as known in the art. Other more specific definitions for certain terms as used in the present application are as set forth below and are intended to apply uniformly throughout the description and claims unless an otherwise expressly set out definition provides a broader definition. Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. Furthermore, the present invention covers all possible combinations of particular and particular embodiments described herein.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The term “about” as used in connection with a numerical value throughout the specification and the claims denotes an interval of accuracy, familiar and acceptable to a person skilled in the art. In general, such interval of accuracy is ±15%. Unless defined otherwise, all technical and scientific terms used herein have the same meaning as commonly understood by one of ordinary skill in the art to which this disclosure is related. Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The invention will be described by way of the following examples which are to be considered as merely illustrative and not limitative of the scope of the invention.

### Examples

### Production and Purification of Omomyc

The Omomyc peptide sequence SEQ ID NO: 4 including a methionine at the N-terminal end was reverse transcribed, codon optimized for expression in E. coli, cloned in a pET3a expression vector (Novagen) and purified from BL21 (DE3) Arabinose-Inducible (Invitrogen®) bacterial strain using protocols adapted from the Max° purification protocol described in J.-F. Naud et al. 2003. J Mol Biol, 326:1577-1595; F.-O. and Mcduff et al. 2009. J Mol Recognit, 22:261-269. The purified construct obtained was the polypeptide of SEQ ID NO: 4. Identity of each purified construct was confirmed by mass spectrometry and by western blot analysis. Omomyc was purified by cationic exchange chromatography and purity was confirmed by mass spectrometry analysis, SDS-PAGE and UV spectroscopy.

Omomyc in Combination with MEK Inhibitors Acts Synergistically in Decreasing the Viability of Melanoma Cancer Cells Two human melanoma cell lines with mutations in BRAF (BRAF^{V600E}) (SkMel28 and SkMel37) were used. Cells were plated in 96 well plates and treated on the following day with increasing concentrations of Trametinib and Omomyc, alone or in combination. After 5 days of treatment, media was removed and AlamarBlue™ Cell Viability Reagent was added to each well, incubated for 3 hours until metabolized, and fluorescence was read with a microplate reader using an excitation between 530-560 and an emission at 590 nm.

Synergy was evaluated using SynergyFinder 3.0 web application derived from Ianevsky A et al. Nucleic Acids Res. 2022; 50(W1):W739-W743.

Synergy scores can be interpreted as the average excess response due to drug interactions. Synergy scores close to 0 give limited confidence in synergy or antagonism. So, when the synergy scores are: less than −10, the interaction between two drugs is likely to be antagonistic. From −10 to 10, the interaction between two drugs is likely to be additive. Greater than 10, the interaction between two drugs is likely to be synergistic.

FIG. 1A shows that in melanoma SkMel28 cells, the treatment with Omomyc or Trametinib reduces the viability of the cells. Treatment of these cells with a combination of Omomyc and Trametinib reveals that the combination of these drugs is synergistic: the ZIP synergy score for the left panel of FIG. 1A is 18.49 and the ZIP synergy score for the right panel of FIG. 1A is 16.87.

FIG. 1B shows that the same is valid for SkMel37 cells. The combination treatment of Omomyc and Trametinib is synergistic: the ZIP synergy score for the left panel of FIG. 1B is 57.54 and the ZIP synergy score for the right panel of FIG. 1B is 46.09.

TABLE 2
Dunnett's multiple comparison test for SkMel37 cells

| Dunnett's multiple | Mean | | |
|---|---|---|---|
| comparisons test | Diff. | 95% CI of diff. | Significant |
| Omomyc 20 μM - Trametinib 0.31 | −39.95 | −62.25 to −17.65 | * |
| nM vs. Omomyc 20 μM | | | |
| Omomyc 20 μM - Trametinib 0.31 | −59.55 | −81.85 to −37.25 | ** |
| nM vs. Trametinib 0.31 nM | | | |
| Omomyc 40 μM - Trametinib 10 | −37.52 | −55.97 to −19.07 | ** |
| nM vs. Omomyc 40 μM | | | |
| Omomyc 40 μM - Trametinib 10 | −60.17 | −76.67 to −43.67 | *** |
| nM vs. Trametinib 10 nM | | | |

The same experiments were carried out with combinations of Omomyc and selumetinib (FIG. 2) or Omomyc and pimasertib (FIG. 3) in SkMel28 and SkMel37 cells revealing that these combinations are also synergistic.

1-15. (canceled)

16. A combination comprising:
i) a first component selected from the group consisting of:
a) a polypeptide comprising the sequence SEQ ID NO: 1 or a functionally equivalent variant thereof;
b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof;
c) a polynucleotide encoding the polypeptide of a) or the conjugate of b);
d) a vector comprising the polynucleotide according to c); and
e) a cell capable of secreting into the medium the polypeptide according to a) or the conjugate according to b);
and
ii) a second component that is a MEK inhibitor, or
a pharmaceutical composition comprising a pharmaceutically effective amount of said combination and a pharmaceutically acceptable excipient.

17. The combination or pharmaceutical composition according to claim 16, wherein the first component is a polypeptide comprising the sequence SEQ ID NO: 1.

18. The combination or pharmaceutical composition according to claim 16, wherein the functionally equivalent variant of SEQ ID NO: 1 is selected from the group consisting of SEQ ID NO: 4, SEQ ID NO: 5, SEQ ID NO; 6, SEQ ID NO: 7, SEQ ID NO: 8, SEQ ID NO: 9, and SEQ ID NO: 10.

19. The combination or pharmaceutical composition according to claim 16, wherein the chemical moiety that facilitates the cellular uptake of the polypeptide or of the functionally equivalent variant thereof is a cell-penetrating peptide sequence and wherein said cell penetrating peptide sequence and said polypeptide or functionally equivalent variant thereof form a fusion protein.

20. The combination or pharmaceutical composition according to claim 16, wherein the conjugate additionally comprises a further nuclear localization signal.

21. The combination or pharmaceutical composition according to claim 16, wherein the MEK inhibitor is selected from the group consisting of trametinib, pimasertib and selumetinib.

22. The combination or pharmaceutical composition according to claim 21, wherein the MEK inhibitor is trametinib.

23. A method for the prevention and/or treatment of cancer that comprises administering to a subject in need thereof a therapeutically effective amount of the combination or pharmaceutical composition according to claim 16.

24. The method according to claim 23, wherein the cancer is selected from the group consisting of melanoma, colorectal cancer and breast cancer.

25. The method according to claim 24, wherein the cancer is melanoma.

26. The method according to claim 23, wherein the cancer is a cancer having a BRAF mutation.

27. The method according to claim 23, wherein the cancer is a cancer resistant to MEK inhibitors.

28. The method according to claim 23, wherein the first component is administered intravenously and the second component is administered orally.


SEQUENCE LISTING (65 sequences)

SEQ ID NO: 1 - protein, 90 aa, synthetic construct
  REGION 1..90: note = Recombinant protein
TEENVKRRTHNVLERQRRNELKRSFFALRDQIPELENNEKAPKVVILKKATAYILSVQAETQKLISEIDLLRKQNEQLKHKLEQLRNSCA

SEQ ID NO: 2 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 3 - protein, 11 aa, synthetic construct
  REGION 1..11: note = Recombinant protein
RQRRNELKRSF

SEQ ID NO: 4 - protein, 91 aa, synthetic construct
  REGION 1..91: note = Recombinant protein
MTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 5 - protein, 90 aa, synthetic construct
  REGION 1..90: note = Recombinant protein
  VARIANT 89: note = X may be any amino acid except C
TEENVKRRTHNVLERQRRNELKRSFFALRDQIPELENNEKAPKVVILKKATAYILSVQAETQKLISEIDLLRKQNEQLKHKLEQLRNSXA

SEQ ID NO: 6 - protein, 91 aa, synthetic construct
  REGION 1..91: note = Recombinant protein
  VARIANT 90: note = X may be any amino acid except C
MTEENVKRRTHNVLERQRRNELKRSFFALRDQIPELENNEKAPKVVILKKATAYILSVQAETQKLISEIDLLRKQNEQLKHKLEQLRNSXA

SEQ ID NO: 7 - protein, 90 aa, synthetic construct
  REGION 1..90: note = Recombinant protein
TEENVKRRTHNVLERQRRNELKRSFFALRDQIPELENNEKAPKVVILKKATAYILSVQAETQKLISEIDLLRKQNEQLKHKLEQLRNSSA

SEQ ID NO: 8 - protein, 91 aa, synthetic construct
  REGION 1..91: note = Recombinant protein
MTEENVKRRTHNVLERQRRNELKRSFFALRDQIPELENNEKAPKVVILKKATAYILSVQAETQKLISEIDLLRKQNEQLKHKLEQLRNSSA

SEQ ID NO: 9 - protein, 90 aa, synthetic construct
  REGION 1..90: note = Recombinant protein
TEENVKRRTHNVLERQRRNELKRSFFALRDQIPELENNEKAPKVVILKKATAYILSVQAETQKLISEIDLLRKQNEQLKHKLEQLRNSAA

SEQ ID NO: 10 - protein, 91 aa, synthetic construct
  REGION 1..91: note = Recombinant protein
MTEENVKRRTHNVLERQRRNELKRSFFALRDQIPELENNEKAPKVVILKKATAYILSVQAETQKLISEIDLLRKQNEQLKHKLEQLRNSAA

SEQ ID NO: 11 - protein, 101 aa, synthetic construct
  REGION 1..101: note = Recombinant protein
MTEENVKRRTHNVLERQRRNELKRSFFALRDQIPELENNEKAPKVVILKKATAYILSVQAETQKLISEIDLLRKQNEQLKHKLEQLRNSCAGRKKRRQRRR

SEQ ID NO: 12 - protein, 99 aa, synthetic construct
  REGION 1..99: note = Recombinant protein
MTEENVKRRTHNVLERQRRNELKRSFFALRDQIPELENNEKAPKVVILKKATAYILSVQAETQKLISEIDLLRKQNEQLKHKLEQLRNSCARRRRRRLR

SEQ ID NO: 13 - protein, 16 aa, synthetic construct
  REGION 1..16: note = CPP sequence found in Drosophila antennapedia protein
RQIKIWFQNRRMKWKK

SEQ ID NO: 14 - protein, 34 aa, synthetic construct
  REGION 1..34: note = CPP sequence found in the herpesvirus simplex 1 (HSV-1) VP22DNA-binding protein
DAATATRGRSAASRPTERPRAPARSASRPRRPVE

SEQ ID NO: 15 - protein, 24 aa, synthetic construct
  REGION 1..24: note = CPPsequence of Bac-7
RRIRPRPPRLPRPRPRPLPFPRPG

SEQ ID NO: 16 - protein, 9 aa, synthetic construct
  REGION 1..9: note = CPP sequence of the HIV-1 TAT protein(amino acids 49-57)
RKKRRQRRR

SEQ ID NO: 17 - protein, 13 aa, synthetic construct
  REGION 1..13: note = CPP sequence of the HIV-1 TAT protein (amino acids 48-60)
GRKKRRQRRRTPQ

SEQ ID NO: 18 - protein, 11 aa, synthetic construct
  REGION 1..11: note = CPP sequence of the HIV-1 TAT protein (amino acids 47-57)
YGRKKRRQRRR

SEQ ID NO: 19 - protein, 20 aa, synthetic construct
  REGION 1..20: note = CPP sequence of S413-PV peptide
ALWKTLLKKVLKAPKKKRKV

SEQ ID NO: 20 - protein, 15 aa, synthetic construct
  REGION 1..15: note = CPP sequence of penetratin
RQIKWFQNRRMKWKK

SEQ ID NO: 21 - protein, 18 aa, synthetic construct
  REGION 1..18: note = CPP sequence of SynB1
RGGRLSYSRRRFSTSTGR

SEQ ID NO: 22 - protein, 10 aa, synthetic construct
  REGION 1..10: note = CPP sequence of SynB3
RRLSYSRRRF

SEQ ID NO: 23 - protein, 12 aa, synthetic construct
  REGION 1..12: note = CPP sequence of PTD-4
PIRRRKKLRRLK

SEQ ID NO: 24 - protein, 12 aa, synthetic construct
  REGION 1..12: note = CPP sequence of PTD-5
RRQRRTSKLMKR

SEQ ID NO: 25 - protein, 15 aa, synthetic construct
  REGION 1..15: note = CPP sequence of the FHV COat (amino acids 35-49)
RRRRNRTRRNRRRVR

SEQ ID NO: 26 - protein, 19 aa, synthetic construct
  REGION 1..19: note = CPP sequence of BMV Gag (amino acids 7-25)
KMTRAQRRAAARRNRWTAR

SEQ ID NO: 27 - protein, 13 aa, synthetic construct
  REGION 1..13: note = CPP sequence of HTLV-II Rex (amino acids 4-16)
TRRQRTRRARRNR

SEQ ID NO: 28 - protein, 13 aa, synthetic construct
  REGION 1..13: note = CPP sequence of D-Tat
GRKKRRQRRRPPQ

SEQ ID NO: 29 - protein, 13 aa, synthetic construct
  REGION 1..13: note = CPP sequence of R9-Tat
GRRRRRRRRRPPQ

SEQ ID NO: 30 - protein, 17 aa, synthetic construct
  REGION 1..17: note = CPP sequence of MAP
KLALKLALKLALALKLA

SEQ ID NO: 31 - protein, 27 aa, synthetic construct
  REGION 1..27: note = CPP sequence of SBP
MGLGLHLLVLAAALQGAWSQPKKKRKV

SEQ ID NO: 32 - protein, 27 aa, synthetic construct
  REGION 1..27: note = CPP sequence of FBP
GALFLGWLGAAGSTMGAWSQPKKKRKV

SEQ ID NO: 33 - protein, 27 aa, synthetic construct
  REGION 1..27: note = CPP sequence of MPG
GALFLGFLGAAGSTMGAWSQPKKKRKV

SEQ ID NO: 34 - protein, 27 aa, synthetic construct
  REGION 1..27: note = CPP sequence of MPG(ENLS)
GALFLGFLGAAGSTMGAWSQPKSKRKV

SEQ ID NO: 35 - protein, 21 aa, synthetic construct
  REGION 1..21: note = CPP sequence of Pep-1
KETWWETWWTEWSQPKKKRKV

SEQ ID NO: 36 - protein, 21 aa, synthetic construct
  REGION 1..21: note = CPP sequence of Pep-2
KETWFETWFTEWSQPKKKRKV

SEQ ID NO: 37 - protein, 10 aa, synthetic construct
  REGION 1..10: note = CPP sequence
GRKKRRQRRR

SEQ ID NO: 38 - protein, 8 aa, synthetic construct
  REGION 1..8: note = CPP sequence
RRRRRRLR

SEQ ID NO: 39 - protein, 12 aa, synthetic construct
  REGION 1..12: note = CPP sequence
RRQRRTSKLMKR

SEQ ID NO: 40 - protein, 27 aa, synthetic construct
  REGION 1..27: note = Transportan peptide
GWTLNSAGYLLGKINLKALAALAKKIL

SEQ ID NO: 41 - protein, 33 aa, synthetic construct
  REGION 1..33: note = CPP sequence
KALAWEAKLAKALAKALAKHLAKALAKALKCEA

SEQ ID NO: 42 - protein, 16 aa, synthetic construct
  REGION 1..16: note = CPP sequence
RQIKIWFQNRRMKWKK

SEQ ID NO: 43 - protein, 11 aa, synthetic construct
  REGION 1..11: note = CPP sequence
YGRKKRRQRRR

SEQ ID NO: 44 - protein, 8 aa, synthetic construct
  REGION 1..8: note = CPP sequence
RKKRRQRR

SEQ ID NO: 45 - protein, 11 aa, synthetic construct
  REGION 1..11: note = CPP sequence
YARAAARQARA

SEQ ID NO: 46 - protein, 11 aa, synthetic construct
  REGION 1..11: note = CPP sequence
THRLPRRRRRR

SEQ ID NO: 47 - protein, 11 aa, synthetic construct
  REGION 1..11: note = CPP sequence
GGRRARRRRRR

SEQ ID NO: 48 - protein, 7 aa, synthetic construct
  REGION 1..7: note = NLS sequence of SV40 large T Antigen
PKKKRKV

SEQ ID NO: 49 - protein, 16 aa, synthetic construct
  REGION 1..16: note = NLS sequence of Nucleoplasmin
KRPAATKKAGQAKKKK

SEQ ID NO: 50 - protein, 18 aa, synthetic construct
  REGION 1..18: note = NLS sequence of CBP80
RRRHSDENDGGQPHKRRK

SEQ ID NO: 51 - protein, 11 aa, synthetic construct
  REGION 1..11: note = NLS sequence of HIV-I Rev protein
RQARRNRRRWE

SEQ ID NO: 52 - protein, 18 aa, synthetic construct
  REGION 1..18: note = NLS sequence of HTLV-I Rex
MPKTRRRPRRSQRKRPPT

SEQ ID NO: 53 - protein, 37 aa, synthetic construct
  REGION 1..37: note = NLS of hnRNP A
NQSSNFGPMKGGNFGGRSSGPYGGGGQYFKPRNQGGY

SEQ ID NO: 54 - protein, 43 aa, synthetic construct
  REGION 1..43: note = NLS of rpL23a
VHSHKKKKIRTSPTFTTPKTLRLRRQPKYPRKSAPRRNKLDHY

SEQ ID NO: 55 - skipped

SEQ ID NO: 56 - protein, 9 aa, synthetic construct
  REGION 1..9: note = M1 peptide (Myc NLS)
PAAKRVKLD

SEQ ID NO: 57 - protein, 11 aa, synthetic construct
  REGION 1..11: note = M2 peptide (Myc NLS)
RQRRNELKRSF

SEQ ID NO: 58 - protein, 6 aa, synthetic construct
  REGION 1..6: note = flexible peptide
GPRRRR

SEQ ID NO: 59 - protein, 8 aa, synthetic construct
  REGION 1..8: note = FLAG-tag
DYKDDDDK

SEQ ID NO: 60 - protein, 8 aa, synthetic construct
  REGION 1..8: note = Strep-tag
WSHPQFEK

SEQ ID NO: 61 - protein, 9 aa, synthetic construct
  REGION 1..9: note = HA-tag
YPYDVPDYA

SEQ ID NO: 62 - protein, 14 aa, synthetic construct
  REGION 1..14: note = V5 tag
GKPIPNPLLGLDST

SEQ ID NO: 63 - protein, 6 aa, synthetic construct
  REGION 1..6: note = Peptide tag
AHGHRP

SEQ ID NO: 64 - protein, 21 aa, synthetic construct
  REGION 1..21: note = Peptide tag
  VARIANT 19: note = X can be any naturally occurring amino acid
  VARIANT 20: note = X can be any naturally occurring amino acid
PIHDHDHPHLVIHSGMTCXXC

SEQ ID NO: 65 - protein, 8 aa, synthetic construct
  REGION 1..8: note = CPP
RRRRRRRR